(19) United States
(12) Patent Application Publication
(10) Pub. No.: US 2011/0263569 A1
(43) Pub. Date: Oct. 27, 2011

(54) RYANODINE CHANNEL BINDERS AND USES THEREOF

(75) Inventor: Elias James Corey, Cambridge, MA (US)

(73) Assignee: President and Fellows of Harvard College

(21) Appl. No.: 12/674,506

(22) PCT Filed: Aug. 21, 2008
(86) PCT No.: PCT/US08/73871

§ 371 Date: May 2, 2011

Related U.S. Application Data
(60) Provisional application No. 60/957,265, filed on Aug. 22, 2007.

(51) Int. Cl. A61K 31/553; A61P 9/00; A61K 31/554; C07D 413/04; C07D 417/04

(52) U.S. Cl. 514/211.09; 540/552

(57) ABSTRACT

Increasing the affinity of calstabin-2 for the cardiac calcium channel RyR2 and thereby stabilizing the channel in the closed state has recently been identified as novel mechanism for treating heart failure, particularly ventricular arrhythmias. JTV-519, a 1,4-benzothiazepine derivative, has been shown to stabilize the calstabin2/RyR2 complex. Novel derivatives of JTV-519 that may be useful in treatment or prevention of heart failure, atrial fibrillation, or exercise-induced cardiac arrhythmias are provided. Synthetic methodology and intermediates in the synthesis of the inventive JTV-519 derivatives are also described.

### RELATED APPLICATIONS

The present application claims priority under 35 U.S.C. §119(e) to U.S. provisional patent application, U.S. Ser. No. 60/957,265, filed Aug. 22, 2007, which is incorporated herein by reference.

### BACKGROUND OF THE INVENTION

JTV-519 is a recently discovered 1,4-benzothiazepine derivatives with antiarrhythmic and cardioprotective properties (FIG. 1) (Wehrens et al., Science 304:292, Apr. 19, 2004; incorporated herein by reference). It functions through stabilizing the ryanodine receptor-calcium release channel in the heart. During diastole, binding of calstabin2 to RyR2 helps to maintain the channel in a closed state to prevent leakage of Ca²⁺ from the sarocoplasmic reticulum into the cytoplasm. In heart failure and catecholaminergic polymorphic ventricular tachycardia, depletion of calstabin2 from the RyR2 macromolecular complex results in leaky RyR2 channels that contribute to both of the diseases (Adam, G.; Andrieux, J.; Plat, M. Tetrahedron 1982, 38, 2403-2410; incorporated herein by reference). Therefore, restoring binding of calstabin2 to RyR2 complex to prevent aberrant Ca²⁺ leakage is a promising approach to preventing arrhythmias.

### SUMMARY OF THE INVENTION

Based on the discovery that stabilizers of the calstabin2/RyR2 complex would be useful in the treatment of arrhythmias, the conformation of JTV-519 was studied under in vivo conditions in hopes of using this information to design new 1,4-benzothiazepine derivatives with better binding affinity for the complex. It is hypothesized that JTV-519 exists as its protonated form in vivo. In order to determine the conformation of JTV-519, the amide side chain of JTV-519 was replaced with a p-nitrobenzoyl group in order to obtain the x-ray crystal structure of compound 2 (FIG. 2). Based on the x-ray structure, various modifications of the structure of JTV-519 were prepared and tested for binding to the calstabin2/RyR2 complex.

In one aspect, the present invention provides derivatives of JTV-519. In certain embodiments, the modification of the JTV-519 structure allows the derivative to prefer the conformation as shown in the x-ray structure of FIG. 2. The derivatives may bind to the calstabin2/RyR2 receptor with greater affinity than the parent compound JTV-519. In certain embodiments, the compound is of the formula:

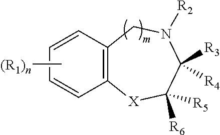

wherein

X is S or O;

m is 0, 1, or 2;

n is an integer between 0 and 4, inclusive;

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —CN; —SCN; —SR_{B}; —SOR_{B}; —SO₂R_{B}; —NO₂; —N₃; —N(R_{B})₂; —NHC(═O)R_{B}; —NR_{B}C(═O)N(R_{B})₂; —OC(═O)OR_{B}; —OC(═O)R_{B}; —OC(═O)N(R_{B})₂; —NR_{B}C(═O)OR_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{S}; —C(═O)R_{C}; —CO₂R_{C}; —CN; —SCN; —SR_{C}; —SOR_{A}; —SO₂R_{C}; —NO₂; —N₃; —N(R_{C})₂; —NHC(═O)R_{C}; —NR_{C}C(═O)N(R_{C})₂; —OC(═O)OR_{C}; —OC(═O)R_{C}; —OC(═O)N(R_{C})₂; —NR_{C}C(═O)OR_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₄ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; —C(═O)R_{D}; —CO₂R_{D}; —CN; —SCN; —SR_{D}; —SOR_{D}; —SO₂R_{D}; —NO₂; —N₃; —N(R_{D})₂; —NHC(═O)R_{D}; —NR_{D}C(═O)N(R_{D})₂; —OC(═O)OR_{D}; —OC(═O)R_{D}; —OC(═O)N(R_{D})₂; —NR_{D}C(═O)OR_{D}; or —C(R_{D})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₅ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{E}; —C(═O)R_{E}; —CO₂R_{E}; —CN; —SCN; —SR_{E}; —SOR_{E}; —SO₂R_{E}; —NO₂; —N₃; —N(R_{E})₂; —NHC(═O)R_{E}; —NR_{E}C(═O)N(R_{E})₂; —OC(═O)OR_{E}; —OC(═O)R_{E}; —OC(═O)N(R_{E})₂; —NR_{E}C(═O)OR_{E}; or —C(R_{E})₃; wherein each occurrence of R_{E} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₆ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{F}; —C(═O)R_{F}; —CO₂R_{F}; —CN; —SCN; —SR_{F}; —SOR_{F}; —SO₂R_{F}; —NO₂; —N₃; —N(R_{F})₂; —NHC(═O)R_{F}; —NR_{F}C(═O)N(R_{F})₂; —OC(═O)OR_{F}; —OC(═O)R_{F}; —OC(═O)N(R_{F})₂; —NR_{F}C(═O)OR_{F}; or —C(R_{F})₃; wherein each occurrence of R_{F} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

In another aspect, the present invention provides a compound of formula:

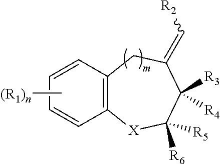

wherein

X is S or O;

m is 0, 1, or 2;

n is an integer between 0 and 4, inclusive;

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —SR_{B}; —SOR_{B}; —SO₂R_{B}; —N(R_{B})₂; —NHC(═O)R_{B}; —NR_{B}C(═O)N(R_{B})₂; —OC(═O)OR_{B}; —OC(═O)R_{B}; —OC(═O)N(R_{B})₂; —NR_{B}C(═O)OR_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —CN; —SCN; —SR_{C}; —SOR_{C}; —SO₂R_{C}; —NO₂; —N₃; —N(R_{C})₂; —NHC(═O)R_{C}; —NR_{C}C(═O)N(R_{C})₂; —OC(═O)OR_{C}; —OC(═O)R_{C}; —OC(═O)N(R_{C})₂; —NR_{C}C(═O)OR_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₄ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; —C(═O)R_{D}; —CO₂R_{D}; —CN; —SCN; —SR_{D}; —SOR_{D}; —SO₂R_{D}; —NO₂; —N₃; —N(R_{D})₂; —NHC(═O)R_{D}; —NR_{D}C(═O)N(R_{D})₂; —OC(═O)OR_{D}; —OC(═O)R_{D}; —OC(═O)N(R_{D})₂; —NR_{D}C(═O)OR_{D}; or —C(R_{D})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₅ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{E}; —C(═O)R_{E}; —CO₂R_{E}; —CN; —SCN; —SR_{E}; —SOR_{E}; —SO₂R_{E}; —NO₂; —N₃; —N(R_{E})₂; —NHC(═O)R_{E}; —NR_{E}C(═O)N(R_{E})₂; —OC(═O)OR_{E}; —OC(═O)R_{E}; —OC(═O)N(R_{E})₂; —NR_{E}C(═O)OR_{E}; or —C(R_{E})₃; wherein each occurrence of R_{E} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₆ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{F}; —C(═O)R_{F}; —CO₂R_{F}; —CN; —SCN; —SR_{F}; —SOR_{F}; —SO₂R_{F}; —NO₂; —N₃; —N(R_{F})₂; —NHC(═O)R_{F}; —NR_{F}C(═O)N(R_{F})₂; —OC(═O)OR_{F}; —OC(═O)R_{F}; —OC(═O)N(R_{F})₂; —NR_{F}C(═O)OR_{F}; or —C(R_{F})₃; wherein each occurrence of R_{F} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

The inventive compound may be prepared by first preparing the 1,4-benzothiazepine heterocycle as shown in the scheme below:

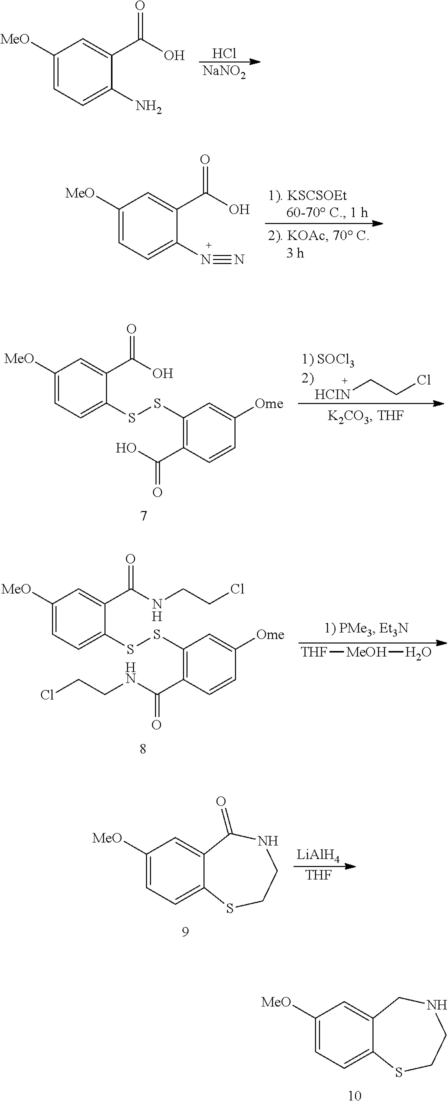

The 1,4-benzothiazepine heterocycle 9 may also be prepared using a Schmidt rearrangement as shown in the schemes below:

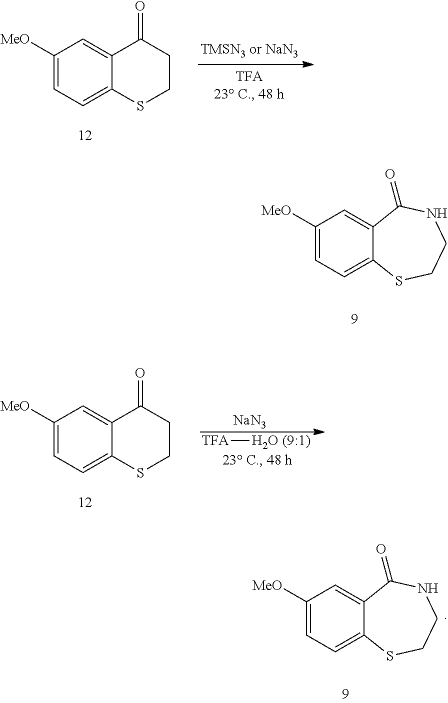

The synthesis of an example of an inventive analogs of JTV-519 (19) is shown in the scheme below:

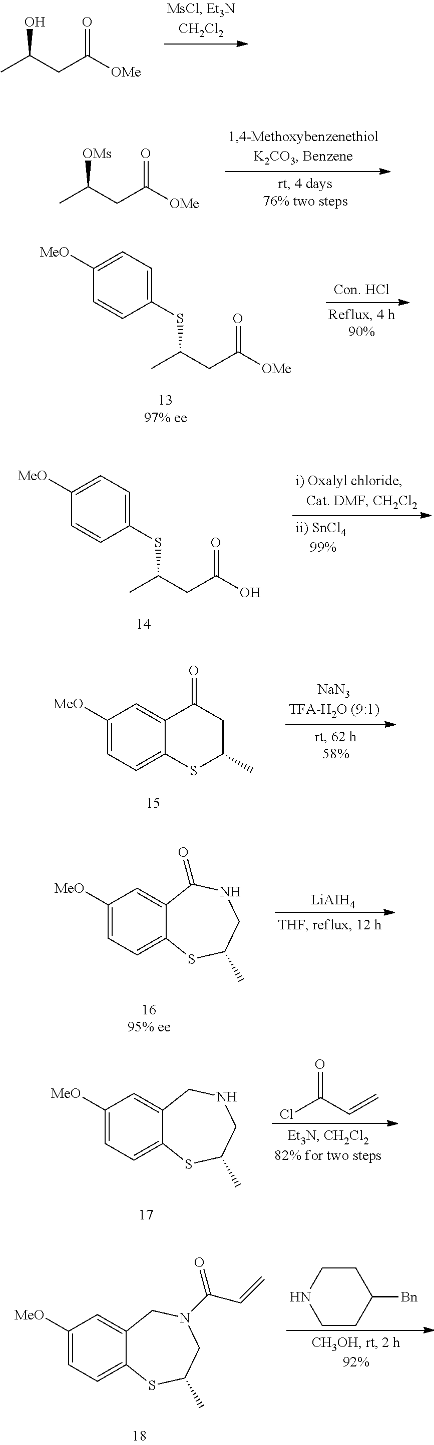

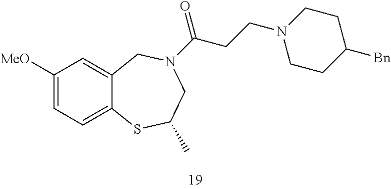

The individual steps as well as the sequence of synthetic steps leading to the inventive compounds are considered to be within the scope of the invention. Intermediates in the synthesis of the inventive compounds are also considered to be within the scope of the invention.

In another aspect, the invention provides pharmaceutical compositions and methods of using the inventive compounds. The pharmaceutical compositions may optionally include a pharmaceutically acceptable excipient. The methods and compositions may be used to treat disease in humans and other animals including domesticated animals. Any mode of administration including oral and parenteral administration of the pharmaceutical composition may be used. The inventive compounds may also be prepared in immediate release formulations, extended release formulations, or controlled release formulations. The compounds are particularly useful in treating a subject with cardiac disease. In certain embodiments, the compounds are used to treat or prevent cardiac arrhythmias (e.g., atrial fribrillation, ventricular arrhythmias, exercise-induced cardiac arrhythmias, catecholaminergic polymorphic ventricular tachycardia). In certain embodiments, the inventive compounds are useful in treating a subject with a cardiac disease associated with a mutation in the ryanodine receptor gene, in particular, RyR2, or in the calstabin2 (FKBP12.6) gene. Without wishing to be bound by any particular theory, the compounds are thought to stabilize the RyR2/calstabin2 (FKBP12.6) complex.

This application refers to various issued patents, published patent applications, journal articles, and other publications, all of which are incorporated herein by reference.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

One of ordinary skill in the art will appreciate that the synthetic methods, as described herein, utilize a variety of protecting groups. By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group should be selectively removable in good yield by readily available, preferably non-toxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Hydroxyl protecting groups include methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolylN-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(NN′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of cardiac disease, particularly heart failure and cardiac arrhythmias. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

In certain embodiments, the alkyl, alkenyl, and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

The term “alkoxy”, or “thioalkyl” as used herein refers to an alkyl group, as previously defined, attached to the parent molecule through an oxygen atom or through a sulfur atom. In certain embodiments, the alkyl, alkenyl, and alkynyl groups contain 1-20 alipahtic carbon atoms. In certain other embodiments, the alkyl, alkenyl, and alkynyl groups contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups contain 1-6 aliphatic carbon atoms. In yet other embodiments, the alkyl, alkenyl, and alkynyl groups contain 1-4 aliphatic carbon atoms. Examples of alkoxy, include but are not limited to, methoxy, ethoxy, propoxy, isopropoxy, n-butoxy, tert-butoxy, neopentoxy, and n-hexoxy. Examples of thioalkyl include, but are not limited to, methylthio, ethylthio, propylthio, isopropylthio, n-butylthio, and the like.

The term “alkylamino” refers to a group having the structure —NHR′, wherein R′ is aliphatic, as defined herein. In certain embodiments, the aliphatic group contains 1-20 aliphatic carbon atoms. In certain other embodiments, the aliphatic group contains 1-10 aliphatic carbon atoms. In yet other embodiments, the aliphatic group employed in the invention contain 1-8 aliphatic carbon atoms. In still other embodiments, the aliphatic group contains 1-6 aliphatic carbon atoms. In yet other embodiments, the aliphatic group contains 1-4 aliphatic carbon atoms. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each an aliphatic group, as defined herein. R and R′ may be the same or different in an dialkyamino moiety. In certain embodiments, the aliphatic groups contains 1-20 aliphatic carbon atoms. In certain other embodiments, the aliphatic groups contains 1-10 aliphatic carbon atoms. In yet other embodiments, the aliphatic groups employed in the invention contain 1-8 aliphatic carbon atoms. In still other embodiments, the aliphatic groups contains 1-6 aliphatic carbon atoms. In yet other embodiments, the aliphatic groups contains 1-4 aliphatic carbon atoms. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of cyclic diaminoalkyl groups include, but are not limited to, aziridinyl, pyrrolidinyl, piperidinyl, morpholinyl, pyrrolyl, imidazolyl, 1,3,4-triazolyl, and tetrazolyl.

Some examples of substituents of the above-described aliphatic (and other) moieties of compounds of the invention include, but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl, and the like. In certain embodiments of the present invention, the term “heteroaryl”, as used herein, refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups can be unsubstituted or substituted, wherein substitution includes replacement of one, two, three, or more of the hydrogen atoms thereon independently with any one or more of the following moieties including, but not limited to: aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic, or heterocyclic moieties, may optionally be substituted with substituents including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “heteroaliphatic”, as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The terms “halo” and “halogen” as used herein refer to an atom selected from fluorine, chlorine, bromine, and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl” or “heterocycle”, as used herein, refers to a non-aromatic 5-, 6-, or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

“Carbocycle”: The term “carbocycle”, as used herein, refers to an aromatic or non-aromatic ring in which each atom of the ring is a carbon atom.

“Independently selected”: The term “independently selected” is used herein to indicate that the R groups can be identical or different.

Definitions of non-chemical terms used throughout the specification include:

“Animal”: The term animal, as used herein, refers to humans as well as non-human animals, at any stage of development, including, for example, mammals, birds, reptiles, amphibians, fish, worms, and single cell organisms. In certain embodiments, the non-human animal is a mammal (e.g., a rodent, a mouse, a rat, a rabbit, a monkey, a dog, a cat, a primate, or a pig). A non-human animal may be a transgenic animal or clone.

“Effective amount”: In general, the “effective amount” of a compound or composition refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient. For example, the effective amount of an inventive compound is the amount that results in prevention of a cardiac arrhythmia. In another example, the effective amount of an inventive compound is the amount sufficient to stabilize the binding of calstabin2 to RyR2 receptor and prevent aberrant Ca²⁺ leakage.

The term “FKBP12.6” and “calstabin2” refers to a FKBP12.6 protein and any analogues thereof as well as polynucleotides that encode any FKBP12.6 protein or analogue thereof FKBP12.6 proteins bind RyR2 protein and stabilize the closed state of the cardiac ryanodine receptor. The binding of FKBP12.6 to RyR2 prevents aberrant activation of the calcium channel during the resting phase of the cardiac cycle. FKBP12.6 analogues may have at least 50%, at least 60%, at least 70%, at least 80%, at least 90%, at least 95%, or at least 98% homology to wild type FKBP12.6. “FKBP12.6” may refer to FKBP12.6 polypeptides, FKBP12.6 proteins, FKBP12.6 peptides, FKBP12.6 fragments, FKBP12.6 variants, and FKBP12.6 mutants thereof as well as to nucleic acids that encode FKBP12.6 polypeptides, FKBP12.6 proteins, FKBP12.6 peptides, FKBP12.6 fragments, FKBP12.6 variants, or FKBP12.6 mutants thereof.

The term “RyR2” refers to a RyR2 protein and any analogues thereof as well as polynucleotides that encode any RyR2 protein or analogue. RyR2 proteins form a calcium channel in the sarcoplasmic reticulum of cardiac mycocytes. The cardiac ryanodine receptor is a protein complex comprising four 565,000-dalton RyR2 proteins in association with four 12,000-dalton FKBP12.6 proteins (also known as calstabin2). RyR2 may be in any form such as phosphorylated, unphosphorylated, or hyperphosphorylated. RyR2 analogues may have at least 50%, at least 60%, at least 70%, at least 80%, at least 90%, at least 95%, or at least 98% homology to wild type RyR2. “RyR2” may refer to RyR2 polypeptides, RyR2 proteins, RyR2 peptides, RyR2 fragments, RyR2 variants, and RyR2 mutants thereof as well as to nucleic acids that encode RyR2 polypeptides, RyR2 proteins, RyR2 peptides, RyR2 fragments, RyR2 variants, or RyR2 mutants thereof.

### BRIEF DESCRIPTION OF THE DRAWING

FIG. 1 shows the chemical structure of JTV-519, which has been shown to have anti-arrhythmic and cardioprotective properties.

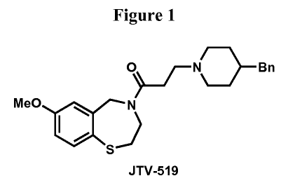

FIG. 2 shows the structure of a p-nitrobenzoyl derivative of JTV-519 and its x-ray structure.

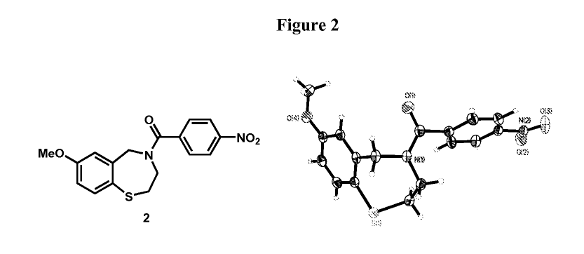

### DETAILED DESCRIPTION OF CERTAIN EMBODIMENTS OF THE INVENTION

The present invention provides novel derivatives of 1,4-benzothiazepine, JTV-519, which is a modulator of calcium ion channels and has been shown previously to exhibit anti-arrhythmic and cardioprotective properties. JTV-519 is thought to increase the affinity of calstabin2 (also known as FKBP12.6) for RyR2, which stabilizes the closed state of the channel and thereby prevents aberrant Ca²⁺ leakage. The present invention also provides pharmaceutical compositions and methods of using the inventive compounds to treat or prevent cardiac disease including heart failure and cardiac arrhythmias. The present invention also provides synthetic methodologies and intermediates for preparing the inventive compounds.

### Compounds

Compounds of the present invention include derivatives of JTV-519 (4-[3-(4-benzylpiperidin-1-yl)propionyl]-7methoxy-2,3,4,5-tetrahydro-1,4-benzothiazepine). Particularly useful compounds of the present invention include those with biological activity, particularly the ability to increase the affinity of calstabin2 for RyR2; stabilize the closed state of the RyR2 channel; and/or prevent the leakage of Ca²⁺ from the sarcoplasmic reticulum through the ryanodine receptor-calcium-release channel (RyR2). Without wishing to be bound by any particular theory, the inventive compounds are thought to enhance the binding of calstabin2 to RyR2. The stabilization of the calstabin2/RyR2 complex prevents the aberrant leakage of Ca²⁺ from the sarcoplasmic reticulum. This leakage from the sarcoplasmic reticulum can generate delayed after-depolarizations that can induce ventricular tachycardia and even lead to sudden death. Chronic leakage can also lead to a reduction in myocardial contractility due to a depletion of calcium stores. The inventive compounds are therefore useful in the treatment of cardiac disease. In particular, the compounds are useful in treating and/or preventing cardiac arrhythmias (e.g., ventricular tachycardia).

In certain embodiments, the compounds of the present invention are represented by the formula:

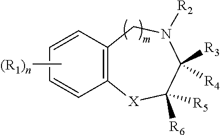

wherein

X is S or O;

m is 0, 1, or 2;

n is an integer between 0 and 4, inclusive;

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —CN; —SCN; —SR_{B}; —SOR_{B}; —SO₂R_{B}; —NO₂; —N₃; —N(R_{B})₂; —NHC(═O)R_{B}; —NR_{B}C(═O)N(R_{B})₂; —OC(═O)OR_{B}; —OC(═O)R_{B}; —OC(═O)N(R_{B})₂; —NR_{B}C(═O)OR_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —CN; —SCN; —SR_{C}; —SOR_{C}; —SO₂R_{C}; —NO₂; —N₃; —N(R_{C})₂; —NHC(═O)R_{C}; —NR_{C}C(═O)N(R_{C})₂; —OC(═O)OR_{C}; —OC(═O)R_{C}; —OC(═O)N(R_{C})₂; —NR_{C}C(═O)OR_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₄ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; —C(═O)R_{D}; —CO₂R_{D}; —CN; —SCN; —SR_{D}); —SOR_{D}; —SO₂R_{D}; —NO₂; —N₃; —N(R_{D})₂; —NHC(═O)R_{D}; —NR_{D}C(═O)N(R_{D})₂; —OC(═O)OR_{D}; —OC(═O)R_{D}; —OC(═O)N(R_{D})₂; —NR_{D}C(═O)OR_{D}; or —C(R_{D})₃; wherein each occurrence of R_{D} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₅ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{E}; —C(═O)R_{E}; —CO₂R_{E}; —CN; —SCN; —SR_{E}; —SOR_{E}; —SO₂R_{E}; —NO₂; —N₃; —N(R_{E})₂; —NHC(═O)R_{E}; —NR_{E}C(═O)N(R_{E})₂; —OC(═O)OR_{E}; —OC(═O)R_{E}; —OC(═O)N(R_{E})₂; —NR_{E}C(═O)OR_{E}; or —C(R_{E})₃; wherein each occurrence of R_{E} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₆ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{F}; —C(═O)R_{F}; —CO₂R_{F}; —CN; —SCN; —SR_{F}; —SOR_{F}; —SO₂R_{F}; —NO₂; —N₃; —N(R_{F})₂; —NHC(═O)R_{F}; —NR_{F}C(═O)N(R_{F})₂; —OC(═O)OR_{F}; —OC(═O)R_{F}; —OC(═O)N(R_{F})₂; —NR_{F}C(═O)OR_{F}; or —C(R_{F})₃; wherein each occurrence of R_{F} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

In certain embodiments, X is S. In certain embodiments, X is O.

In certain embodiments, n is 0. In certain embodiments, n is 1. In certain embodiments, n is 2. In certain embodiments, n is 3. In certain embodiments, n is 4.

In certain embodiments, m is 0. In certain embodiments, m is 1. In certain embodiments, m is 2. In certain embodiments, X is S, and m is 1. In certain embodiments, X is O, and m is 1.

In certain embodiments, R₁ is a halogen. In certain embodiments, R₁ is a substituted or unsubstituted, branched or unbranched aliphatic moiety. In certain embodiments, R₁ is an alkyl moiety. In certain embodiments, R₁ is C₁-C₆ alkyl. In certain embodiments, R₁ is a substituted or unsubstituted, branched or unbranched heteroaliphatic moiety. In certain embodiments, R₁ is substituted or unsubstituted, branched or unbranched acyl. In certain embodiments, R₁ is substituted or unsubstituted, branched or unbranched aryl. In certain embodiments, R₁ is substituted or unsubstituted, branched or unbranched heteroaryl. In certain embodiments, R₁ is —OR_{A}. In certain embodiments, R₁ is —OR_{A},

wherein R_{A} is C₁-C₆ alkyl. In certain embodiments, R₁ is —OMe. In certain embodiments, R₁ is —SR_{A}. In certain embodiments, R₁ is —SR_{A}, wherein R_{A} is C₁-C₆ alkyl. In certain embodiments, R₁ is —N(R_{A})₂.

In certain embodiments, R₂ is hydrogen. In certain embodiments, R₂ is a substituted or unsubstituted, branched or unbranched aliphatic moiety. In certain embodiments, R₂ is an alkyl moiety. In certain embodiments, R₂ is C₁-C₆ alkyl. In certain embodiments, R₂ is a substituted or unsubstituted, branched or unbranched heteroaliphatic moiety. In certain embodiments, R₂ is substituted or unsubstituted, branched or unbranched acyl. In certain embodiments, R₂ is substituted or unsubstituted, branched or unbranched aryl. In certain embodiments, R₂ is substituted or unsubstituted, branched or unbranched heteroaryl. In certain embodiments, R₂ is —C(═O)R_{B}. In certain embodiments, R₂ is —C(═O)R_{B}, wherein R_{B} is substituted or unsubstituted, cyclic heteroaliphatic. In certain embodiments, R₂ is —C(═O)R_{B}, wherein R_{B} is substituted or unsubstituted, cyclic heteroaliphaticalkyl. In certain embodiments, R₂ is —C(═O)OR_{B}. In certain embodiments, R₂ is —C(═O)N(R_{B})₂. In certain embodiments, R₂ is

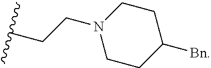

In certain embodiments, R₃ is hydrogen. In certain embodiments, R₃ is a halogen. In certain embodiments, R₃ is a substituted or unsubstituted, branched or unbranched aliphatic moiety. In certain embodiments, R₃ is an alkyl moiety. In certain embodiments, R₃ is C₁-C₆ alkyl. In certain embodiments, R₃ is methyl. In certain embodiments, R₃ is ethyl. In certain embodiments, R₃ is propyl. In certain embodiments, R₃ is iso-propyl. In certain embodiments, R₃ is a substituted or unsubstituted, branched or unbranched heteroaliphatic moiety. In certain embodiments, R₃ is substituted or unsubstituted, branched or unbranched acyl. In certain embodiments, R₃ is substituted or unsubstituted, branched or unbranched aryl. In certain embodiments, R₃ is substituted or unsubstituted, branched or unbranched heteroaryl. In certain embodiments, R₃ is —OR_{C}. In certain embodiments, R₃ is —OR_{C}, wherein R_{C} is C₁-C₆ alkyl. In certain embodiments, R₃ is —OMe. In certain embodiments, R₃ is —SR_{C}. In certain embodiments, R₃ is —SR_{C}, wherein R_{C} is C₁-C₆ alkyl. In certain embodiments, R₃ is —SMe. In certain embodiments, R₃ is —N(R_{C})₂. In certain embodiments, R₃ is —NHR_{C}. In certain embodiments, R₃ is —NH₂.

In certain embodiments, R₄ is hydrogen. In certain embodiments, R₄ is a halogen. In certain embodiments, R₄ is a substituted or unsubstituted, branched or unbranched aliphatic moiety. In certain embodiments, R₄ is an alkyl moiety. In certain embodiments, R₄ is C₁-C₆ alkyl. In certain embodiments, R₄ is methyl. In certain embodiments, R₄ is ethyl. In certain embodiments, R₄ is propyl. In certain embodiments, R₄ is iso-propyl. In certain embodiments, R₄ is a substituted or unsubstituted, branched or unbranched heteroaliphatic moiety. In certain embodiments, R₄ is substituted or unsubstituted, branched or unbranched acyl. In certain embodiments, R₄ is substituted or unsubstituted, branched or unbranched aryl. In certain embodiments, R₄ is substituted or unsubstituted, branched or unbranched heteroaryl. In certain embodiments, R₄ is —OR_{D}. In certain embodiments, R₄ is —OR_{D}, wherein R_{D} is C₁-C₆ alkyl. In certain embodiments, R₄ is —OMe. In certain embodiments, R₄ is —SR_{D}. In certain embodiments, R₄ is —SR_{D}, wherein R_{D} is C₁-C₆ alkyl. In certain embodiments, R₄ is —SMe. In certain embodiments, R₄ is —N(R_{D})₂. In certain embodiments, R₄ is —NHR_{D}. In certain embodiments, R₄ is —NH₂.

In certain embodiments, R₅ is hydrogen. In certain embodiments, R₅ is a halogen. In certain embodiments, R₅ is a substituted or unsubstituted, branched or unbranched aliphatic moiety. In certain embodiments, R₅ is an alkyl moiety. In certain embodiments, R₅ is C₁-C₆ alkyl. In certain embodiments, R₅ is methyl. In certain embodiments, R₅ is ethyl. In certain embodiments, R₅ is propyl. In certain embodiments, R₅ is iso-propyl. In certain embodiments, R₅ is a substituted or unsubstituted, branched or unbranched heteroaliphatic moiety. In certain embodiments, R₅ is substituted or unsubstituted, branched or unbranched acyl. In certain embodiments, R₅ is substituted or unsubstituted, branched or unbranched aryl. In certain embodiments, R₅ is substituted or unsubstituted, branched or unbranched heteroaryl. In certain embodiments, R₅ is —OR_{E}. In certain embodiments, R₅ is —OR_{E}, wherein R_{E} is C₁-C₆ alkyl. In certain embodiments, R₅ is —OMe. In certain embodiments, R₅ is —SR_{E}. In certain embodiments, R₅ is —SR_{E}, wherein R_{E} is C₁-C₆ alkyl. In certain embodiments, R₅ is —SMe. In certain embodiments, R₅ is —N(R_{E})₂. In certain embodiments, R₅ is —NHR_{E}. In certain embodiments, R₅ is —NH₂.

In certain embodiments, R₆ is hydrogen. In certain embodiments, R₆ is a halogen. In certain embodiments, R₆ is a substituted or unsubstituted, branched or unbranched aliphatic moiety. In certain embodiments, R₆ is an alkyl moiety. In certain embodiments, R₆ is C₁-C₆ alkyl. In certain embodiments, R₆ is methyl. In certain embodiments, R₆ is ethyl. In certain embodiments, R₆ is propyl. In certain embodiments, R₆ is iso-propyl. In certain embodiments, R₆ is a substituted or unsubstituted, branched or unbranched heteroaliphatic moiety. In certain embodiments, R₆ is substituted or unsubstituted, branched or unbranched acyl. In certain embodiments, R₆ is substituted or unsubstituted, branched or unbranched aryl. In certain embodiments, R₆ is substituted or unsubstituted, branched or unbranched heteroaryl. In certain embodiments, R₆ is —OR_{F}. In certain embodiments, R₆ is —OR_{F}, wherein R_{F} is C₁-C₆ alkyl. In certain embodiments, R₆ is —OMe. In certain embodiments, R₆ is —SR_{F}. In certain embodiments, R₆ is —SR_{F}, wherein R_{F} is C₁-C₆ alkyl. In certain embodiments, R₆ is —SMe. In certain embodiments, R₆ is —N(R_{F})₂. In certain embodiments, R₆ is —NHR_{F}. In certain embodiments, R₆ is —NH₂.

In certain embodiments, R₃ is not hydrogen; and R₄, R₅, and R₆ are all hydrogen. In certain embodiments, R₃ is C₁-C₆ alkyl; and R₄, R₅, and R₆ are all hydrogen. In certain embodiments, R₃ is methyl; and R₄, R₅, and R₆ are all hydrogen. In certain embodiments, R₄ is not hydrogen; and R₃, R₅, and R₆ are all hydrogen. In certain embodiments, R₄ is C₁-C₆ alkyl; and R₃, R₅, and R₆ are all hydrogen. In certain embodiments, R₄ is methyl; and R₃, R₅, and R₆ are all hydrogen. In certain embodiments, R₅ is not hydrogen; and R₃, R₄, and R₆ are all hydrogen. In certain embodiments, R₅ is C₁-C₆ alkyl; and R₃, R₄, and R₆ are all hydrogen. In certain embodiments, R₅ is methyl; and R₃, R₄, and R₆ are all hydrogen. In certain embodiments, R₆ is not hydrogen; and R₃, R₄, and R₅ are all hydrogen. In certain embodiments, R₆ is C₁-C₆ alkyl; and R₃, R₄, and R₅ are all hydrogen. In certain embodiments, R₆ is methyl; and R₃, R₄, and R₅ are all hydrogen.

In certain embodiments, at least one of R₃, R₄, R₅, and R₆ is not hydrogen. In certain embodiments, at least one of R₃, R₄, R₅, and R₆ is C₁-C₆ alkyl. In certain embodiments, at least one of R₃, R₄, R₅, and R₆ is methyl.

In certain embodiments, R₃ and R₆ are not hydrogen; and R₄ and R₅ are hydrogen. In certain embodiments, R₃ and R₆ are C₁-C₆ alkyl; and R₄ and R₅ are hydrogen. In certain embodiments, R₃ and R₆ are methyl; and R₄ and R₅ are hydrogen.

In certain embodiments, R₄ and R₅ are not hydrogen; and R₃ and R₆ are hydrogen. In certain embodiments, R₄ and R₅ are C₁-C₆ alkyl; and R₃ and R₆ are hydrogen. In certain embodiments, R₄ and R₅ are methyl; and R₃ and R₆ are hydrogen.

In certain embodiments, R₃ and R₅ are not hydrogen; and R₄ and R₆ are hydrogen. In certain embodiments, R₃ and R₅ are C₁-C₆ alkyl; and R₄ and R₆ are hydrogen. In certain embodiments, R₃ and R₅ are methyl; and R₄ and R₆ are hydrogen.

In certain embodiments, R₄ and R₆ are not hydrogen; and R₃ and R₅ are hydrogen. In certain embodiments, R₄ and R₆ are C₁-C₆ alkyl; and R₃ and R₅ are hydrogen. In certain embodiments, R₄ and R₆ are methyl; and R₃ and R₅ are hydrogen.

In certain embodiments, the compound is of formula:

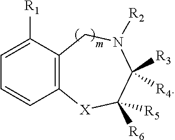

In certain embodiments, the compound is of formula:

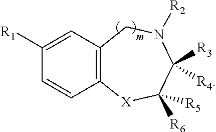

In certain embodiments, the compound is of formula:

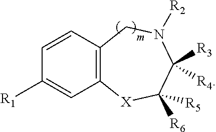

In certain embodiments, the compound is of formula:

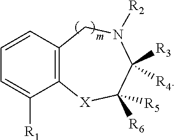

In certain embodiments, the compound is of formula:

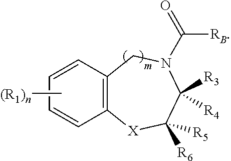

In certain embodiments, the compound is of formula:

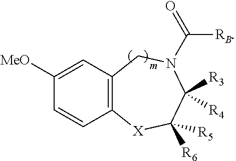

In certain embodiments, the compound is of formula:

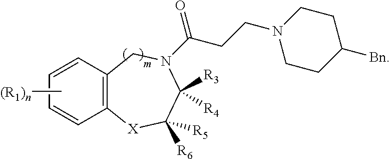

In certain embodiments, the compound of the invention is not of formula:

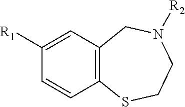

wherein R₁ is —OMe; and R₂ is —C(═O)R_{B} or —SO₂R_{B}.

In certain embodiments, the compounds of the invention do not include the compounds described in published U.S. patent application, US 2005/0187386, published Aug. 25, 2005; which is incorporated herein by reference.

Exemplary compound of the invention include:

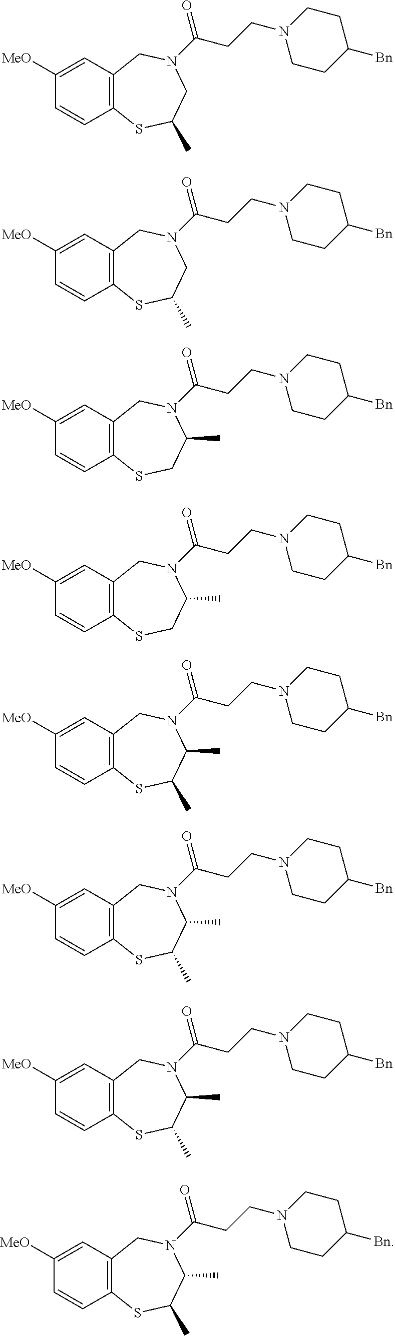

Exemplary compound of the invention include:

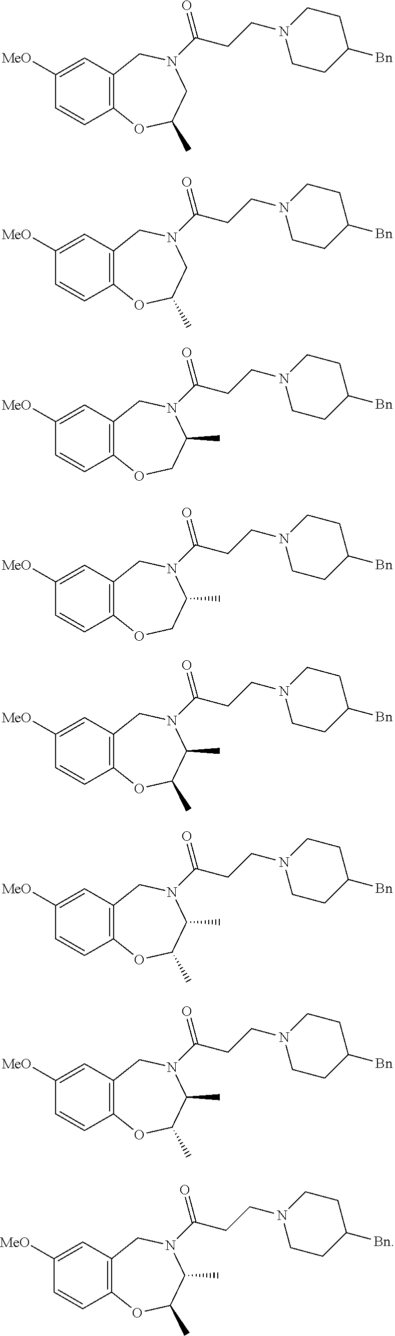

In certain embodiments, the compounds of the present invention are represented by the formula:

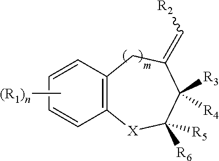

wherein

X is S or O;

m is 0, 1, or 2;

n is an integer between 0 and 4, inclusive;

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —SR_{B}; —SOR_{B}; —SO₂R_{B}; —N(R_{B})₂; —NHC(═O)R_{B}; —NR_{B}C(═O)N(R_{B})₂; —OC(═O)OR_{B}; —OC(═O)R_{B}; —OC(═O)N(R_{B})₂; —NR_{B}C(═O)OR_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —CN; —SCN; —SR_{C}; —SOR_{C}; —SO₂R_{C}; —NO₂; —N₃; —N(R_{C})₂; —NHC(═O)R_{C}; —NR_{C}C(═O)N(R_{C})₂; —OC(═O)OR_{C}; —OC(═O)R_{C}; —OC(═O)N(R_{C})₂; —NR_{C}C(═O)OR_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₄ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; —C(═O)R_{D}; —CO₂R_{D}; —CN; —SCN; —SR_{C}; —SOR_{D}; —SO₂R_{D}; —NO₂; —N₃; —N(R_{D})₂; —NHC(═O)R_{D}; —NR_{D}C(═O)N(R_{D})₂; —OC(═O)OR_{D}; —OC(═O)R_{D}; —OC(═O)N(R_{D})₂; —NR_{D}C(═O)OR_{D}; or —C(R_{D})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₅ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{E}; —C(═O)R_{E}; —CO₂R_{E}; —CN; —SCN; —SR_{E}; —SOR_{E}; —SO₂R_{E}; —NO₂; —N₃; —N(R_{E})₂; —NHC(═O)R_{E}; —NR_{E}C(═O)N(R_{E})₂; —OC(═O)OR_{E}; —OC(═O)R_{E}; —OC(═O)N(R_{E})₂; —NR_{E}C(═O)OR_{E}; or —C(R_{E})₃; wherein each occurrence of R_{E} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₆ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{F}; —C(═O)R_{F}; —CO₂R_{F}; —CN; —SCN; —SR_{F}; —SOR_{F}; —SO₂R_{F}; —NO₂; —N₃; —N(R_{F})₂; —NHC(═O)R_{F}; —NR_{F}C(═O)N(R_{F})₂; —OC(═O)OR_{F}; —OC(═O)R_{F}; —OC(═O)N(R_{F})₂; —NR_{F}C(═O)OR_{F}; or —C(R_{F})₃; wherein each occurrence of R_{F} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

In certain embodiments, the compound is of formula:

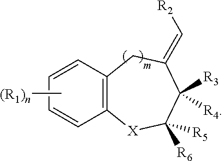

In certain embodiments, the compound is of formula:

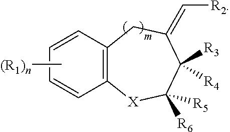

In certain embodiments, X is S. In certain embodiments, X is O.

In certain embodiments, n is 0. In certain embodiments, n is 1. In certain embodiments, n is 2. In certain embodiments, n is 3. In certain embodiments, n is 4.

In certain embodiments, m is 0. In certain embodiments, m is 1. In certain embodiments, m is 2. In certain embodiments, X is S, and m is 1. In certain embodiments, X is O, and m is 1.

In certain embodiments, R₁ is a halogen. In certain embodiments, R₁ is a substituted or unsubstituted, branched or unbranched aliphatic moiety. In certain embodiments, R₁ is an alkyl moiety. In certain embodiments, R₁ is C₁-C₆ alkyl. In certain embodiments, R₁ is a substituted or unsubstituted, branched or unbranched heteroaliphatic moiety. In certain embodiments, R₁ is substituted or unsubstituted, branched or unbranched acyl. In certain embodiments, R₁ is substituted or unsubstituted, branched or unbranched aryl. In certain embodiments, R₁ is substituted or unsubstituted, branched or unbranched heteroaryl. In certain embodiments, R₁ is —OR_{A}. In certain embodiments, R₁ is —OR_{A}, wherein R_{A} is C₁-C₆ alkyl. In certain embodiments, R₁ is —OMe. In certain embodiments, R₁ is —SR_{A}. In certain embodiments, R₁ is —SR_{A}, wherein R_{A} is C₁-C₆ alkyl. In certain embodiments, R₁ is —N(R_{A})₂.

In certain embodiments, R₂ is hydrogen. In certain embodiments, R₂ is a substituted or unsubstituted, branched or unbranched aliphatic moiety. In certain embodiments, R₂ is an alkyl moiety. In certain embodiments, R₂ is C₁-C₆ alkyl. In certain embodiments, R₂ is a substituted or unsubstituted, branched or unbranched heteroaliphatic moiety. In certain embodiments, R₂ is substituted or unsubstituted, branched or unbranched acyl. In certain embodiments, R₂ is substituted or unsubstituted, branched or unbranched aryl. In certain embodiments, R₂ is substituted or unsubstituted, branched or unbranched heteroaryl. In certain embodiments, R₂ is —C(═O)R_{B}. In certain embodiments, R₂ is —C(═O)R_{B}, wherein R_{B} is substituted or unsubstituted, cyclic heteroaliphatic. In certain embodiments, R₂ is —C(═O)R_{B}, wherein R_{B} is substituted or unsubstituted, cyclic heteroaliphaticalkyl. In certain embodiments, R₂ is —C(═O)OR_{B}. In certain embodiments, R₂is —C(═O)N(R_{B})₂. In certain embodiments, R₂ is

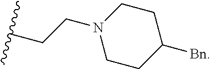

In certain embodiments, R₃ is hydrogen. In certain embodiments, R₃ is a halogen. In certain embodiments, R₃ is a substituted or unsubstituted, branched or unbranched aliphatic moiety. In certain embodiments, R₃ is an alkyl moiety. In certain embodiments, R₃ is C₁-C₆ alkyl. In certain embodiments, R₃ is methyl. In certain embodiments, R₃ is ethyl. In certain embodiments, R₃ is propyl. In certain embodiments, R₃ is iso-propyl. In certain embodiments, R₃ is a substituted or unsubstituted, branched or unbranched heteroaliphatic moiety. In certain embodiments, R₃ is substituted or unsubstituted, branched or unbranched acyl. In certain embodiments, R₃ is substituted or unsubstituted, branched or unbranched aryl. In certain embodiments, R₃ is substituted or unsubstituted, branched or unbranched heteroaryl. In certain embodiments, R₃ is —OR_{C}. In certain embodiments, R₃ is —OR_{S}, wherein R_{C} is C₁-C₆ alkyl. In certain embodiments, R₃ is —OMe. In certain embodiments, R₃ is —SR_{C}. In certain embodiments, R₃ is —SR_{C}, wherein R_{C} is C₁-C₆ alkyl. In certain embodiments, R₃ is —SMe. In certain embodiments, R₃ is —N(R_{C})₂. In certain embodiments, R₃ is —NHR_{C}. In certain embodiments, R₃ is —NH₂.

In certain embodiments, R₄ is hydrogen. In certain embodiments, R₄ is a halogen. In certain embodiments, R₄ is a substituted or unsubstituted, branched or unbranched aliphatic moiety. In certain embodiments, R₄ is an alkyl moiety. In certain embodiments, R₄ is C₁-C₆ alkyl. In certain embodiments, R₄ is methyl. In certain embodiments, R₄ is ethyl. In certain embodiments, R₄ is propyl. In certain embodiments, R₄ is iso-propyl. In certain embodiments, R₄ is a substituted or unsubstituted, branched or unbranched heteroaliphatic moiety. In certain embodiments, R₄ is substituted or unsubstituted, branched or unbranched acyl. In certain embodiments, R₄ is substituted or unsubstituted, branched or unbranched aryl. In certain embodiments, R₄ is substituted or unsubstituted, branched or unbranched heteroaryl. In certain embodiments, R₄ is —OR_{D}. In certain embodiments, R₄ is —OR_{D}, wherein R_{D} is C₁-C₆ alkyl. In certain embodiments, R₄ is —OMe. In certain embodiments, R₄ is —SR_{D}. In certain embodiments, R₄ is —SR_{D}, wherein

R_{D} is C₁-C₆ alkyl. In certain embodiments, R₄ is —SMe. In certain embodiments, R₄ is —N(R_{D})₂. In certain embodiments, R₄ is —NHR_{D}. In certain embodiments, R₄ is —NH₂.

In certain embodiments, R₅ is hydrogen. In certain embodiments, R₅ is a halogen. In certain embodiments, R₅ is a substituted or unsubstituted, branched or unbranched aliphatic moiety. In certain embodiments, R₅ is an alkyl moiety. In certain embodiments, R₅ is C₁-C₆ alkyl. In certain embodiments, R₅ is methyl. In certain embodiments, R₅ is ethyl. In certain embodiments, R₅ is propyl. In certain embodiments, R₅ is iso-propyl. In certain embodiments, R₅ is a substituted or unsubstituted, branched or unbranched heteroaliphatic moiety. In certain embodiments, R₅ is substituted or unsubstituted, branched or unbranched acyl. In certain embodiments, R₅ is substituted or unsubstituted, branched or unbranched aryl. In certain embodiments, R₅ is substituted or unsubstituted, branched or unbranched heteroaryl. In certain embodiments, R₅ is —OR_{E}. In certain embodiments, R₅ is —OR_{E}, wherein R_{E} is C₁-C₆ alkyl. In certain embodiments, R₅ is —OMe. In certain embodiments, R₅ is —SR_{E}. In certain embodiments, R₅ is —SR_{E}, wherein R_{E} is C₁-C₆ alkyl. In certain embodiments, R₅ is —SMe. In certain embodiments, R₅ is —N(R_{E})₂. In certain embodiments, R₅ is —NHR_{E}. In certain embodiments, R₅ is —NH₂.

In certain embodiments, R₆ is hydrogen. In certain embodiments, R₆ is a halogen. In certain embodiments, R₆ is a substituted or unsubstituted, branched or unbranched aliphatic moiety. In certain embodiments, R₆ is an alkyl moiety. In certain embodiments, R₆ is C₁-C₆ alkyl. In certain embodiments, R₆ is methyl. In certain embodiments, R₆ is ethyl. In certain embodiments, R₆ is propyl. In certain embodiments, R₆ is iso-propyl. In certain embodiments, R₆ is a substituted or unsubstituted, branched or unbranched heteroaliphatic moiety. In certain embodiments, R₆ is substituted or unsubstituted, branched or unbranched acyl. In certain embodiments, R₆ is substituted or unsubstituted, branched or unbranched aryl. In certain embodiments, R₆ is substituted or unsubstituted, branched or unbranched heteroaryl. In certain embodiments, R₆ is —OR_{F}. In certain embodiments, R₆ is —OR_{F}, wherein R_{F} is C₁-C₆ alkyl. In certain embodiments, R₆ is —OMe. In certain embodiments, R₆ is —SR_{F}. In certain embodiments, R₆ is —SR_{F}, wherein R_{F} is C₁-C₆ alkyl. In certain embodiments, R₆ is —SMe. In certain embodiments, R₆ is —N(R_{F})₂. In certain embodiments, R₆ is —NHR_{F}. In certain embodiments, R₆ is —NH₂.

In certain embodiments, R₃ is not hydrogen; and R₄, R₅, and R₆ are all hydrogen. In certain embodiments, R₃ is C₁-C₆ alkyl; and R₄, R₅, and R₆ are all hydrogen. In certain embodiments, R₃ is methyl; and R₄, R₅, and R₆ are all hydrogen. In certain embodiments, R₄ is not hydrogen; and R₃, R₅, and R₆ are all hydrogen. In certain embodiments, R₄ is C₁-C₆ alkyl; and R₃, R₅, and R₆ are all hydrogen. In certain embodiments, R₄ is methyl; and R₃, R₅, and R₆ are all hydrogen. In certain embodiments, R₅ is not hydrogen; and R₃, R₄, and R₆ are all hydrogen. In certain embodiments, R₅ is C₁-C₆ alkyl; and R₃, R₄, and R₆ are all hydrogen. In certain embodiments, R₅ is methyl; and R₃, R₄, and R₆ are all hydrogen. In certain embodiments, R₆ is not hydrogen; and R₃, R₄, and R₅ are all hydrogen. In certain embodiments, R₆ is C₁-C₆ alkyl; and R₃, R₄, and R₅ are all hydrogen. In certain embodiments, R₆ is methyl; and R₃, R₄, and R₅ are all hydrogen.

In certain embodiments, R₃ and R₆ are not hydrogen; and R₄ and R₅ are hydrogen. In certain embodiments, R₃ and R₆ are C₁-C₆ alkyl; and R₄ and R₅ are hydrogen. In certain embodiments, R₃ and R₆ are methyl; and R₄ and R₅ are hydrogen.

In certain embodiments, R₄ and R₅ are not hydrogen; and R₃ and R₆ are hydrogen. In certain embodiments, R₄ and R₅ are C₁-C₆ alkyl; and R₃ and R₆ are hydrogen. In certain embodiments, R₄ and R₅ are methyl; and R₃ and R₆ are hydrogen.

In certain embodiments, R₃ and R₅ are not hydrogen; and R₄ and R₆ are hydrogen. In certain embodiments, R₃ and R₅ are C₁-C₆ alkyl; and R₄ and R₆ are hydrogen. In certain embodiments, R₃ and R₅ are methyl; and R₄ and R₆ are hydrogen.

In certain embodiments, R₄ and R₆ are not hydrogen; and R₃ and R₅ are hydrogen. In certain embodiments, R₄ and R₆ are C₁-C₆ alkyl; and R₃ and R₅ are hydrogen. In certain embodiments, R₄ and R₆ are methyl; and R₃ and R₅ are hydrogen.

Exemplary compound of the invention include:

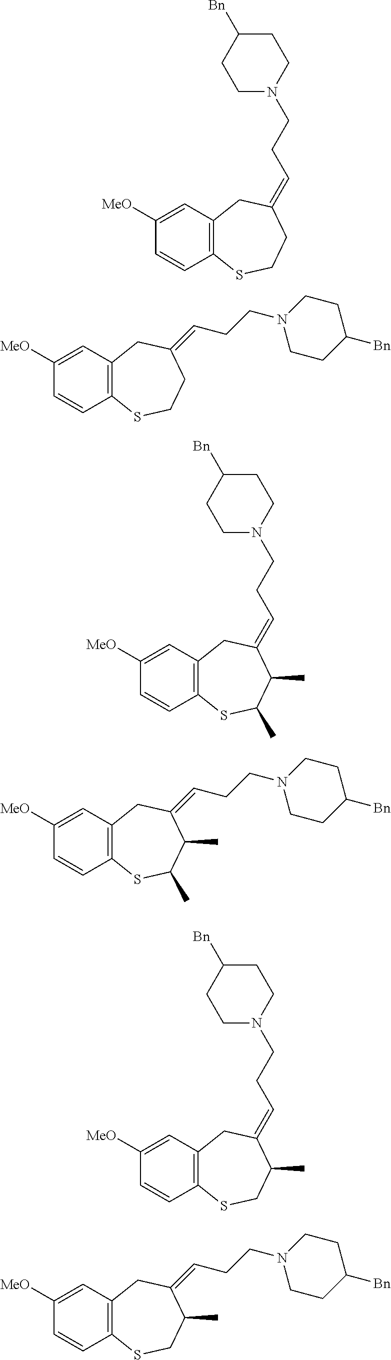

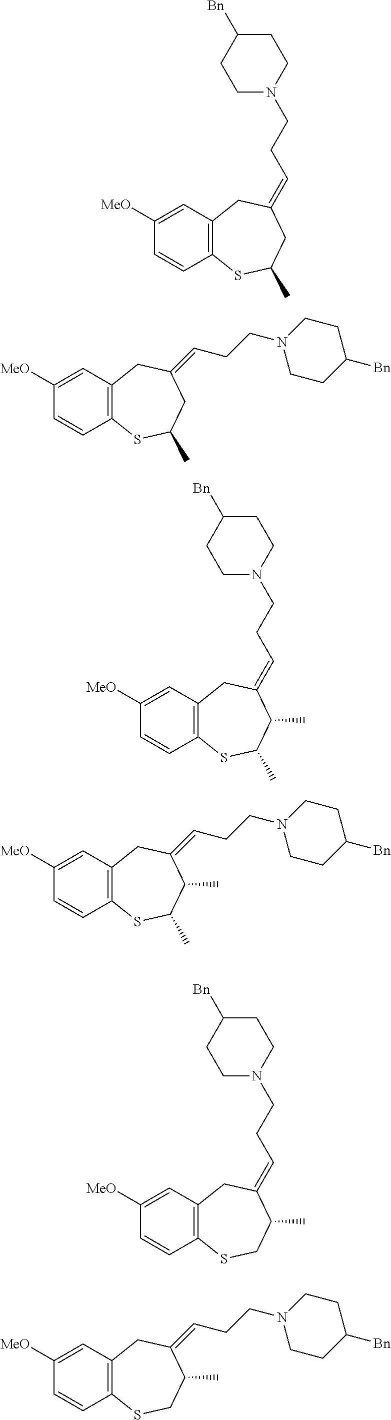

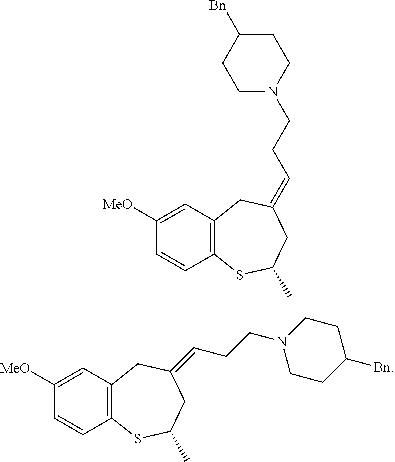

### Methods of Synthesis

The present invention also includes all steps and methodologies used in preparing the compounds of the invention as well as intermediates along the synthetic route. An exemplary synthesis of an inventive compound is shown below:

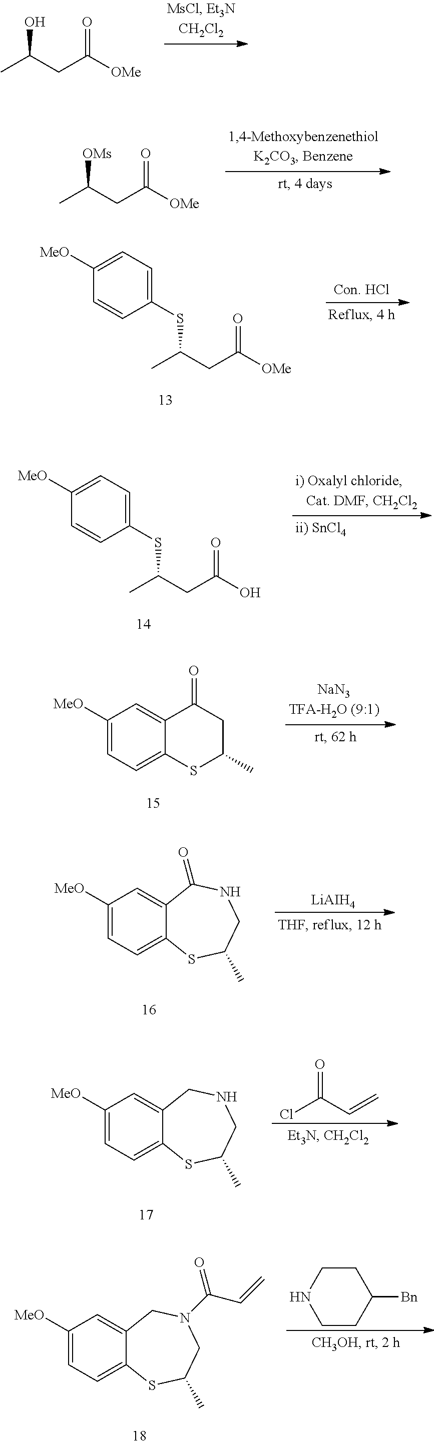

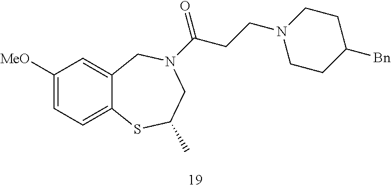

As would be appreciated by one of skill in the art, the synthetic methodology may be used to vary the substituents on the phenyl ring or 7-membered ring. Derivatization of the 7-membered ring may be useful in holding the ring system in a particular conformation for binding and/or stabilizing the ryanodine receptor.

In certain embodiments, the invention provides a method of preparing a 1,4-benzothiazepine intermediate of formula:

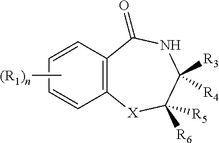

wherein R₁, R₃, R₄, R₅, R₆, X, and n are defined herein, comprising reacting a ketone of formula:

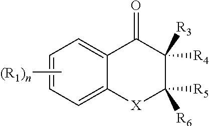

with NaN₃ in a TFA-H₂O solvent system. In certain embodiments, the TFA-H₂O solvent system comprises approximately 10 parts TFA to approximately 1 part water. In certain embodiments, the TFA-H₂O solvent system comprises approximately 9 parts TFA to approximately 1 part water. In certain embodiments, the TFA-H₂O solvent system comprises approximately 8 parts TFA to approximately 2 parts water.

In certain embodiments, the invention provides an intermediate of formula:

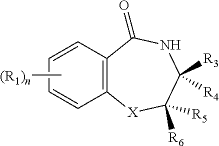

wherein R₁, R₃, R₄, R₅, R₆, X, and n are defined herein.

In certain embodiments, the invention provides a method of preparing a 1,4-benzothiazepine of formula:

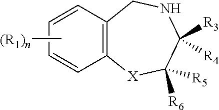

wherein R₁, R₃, R₄, R₅, R₆, R_{B}, X, and n are defined herein, comprising reducing an amide of formula:

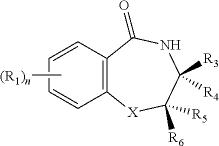

under suitable conditions to form a 1,4-benzothiazepine of formula:

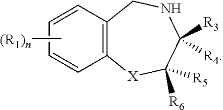

In certain embodiments, the invention provides an intermediate of formula:

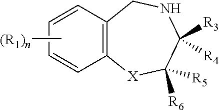

wherein R₁, R₃, R₄, R₅, R₆, X, and n are defined herein.

In certain embodiments, the invention provides a method of preparing a 1,4-benzothiazepine of formula:

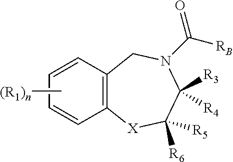

wherein R₁, R₃, R₄, R₅, R₆, R_{B}, X, and n are defined herein, comprising reacting an amine of formula:

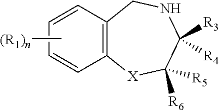

with an acyl donor under suitable conditions to form a compound of formula:

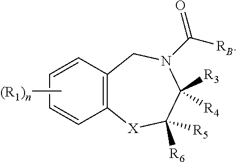

In certain embodiments, the acyl donor is an acyl chloride.

Various substituents of the 1,4-benzothiazepine may be derivatized, transformed, or substituted using synthetic methods known in the art.

As will be appreciated by one of skill in the art, various isolation and purification techniques including flash chromatography, crystallization, distillation, HPLC, thin layer chromatography, extraction, filtration, etc. may be used in the course of synthesizing compounds of the invention. These techniques may be used in the preparation or purification of intermediates, reagents, products, starting materials, or solvents.

### Uses of Compounds and Pharmaceutical Compositions

The invention provides methods of using the inventive compounds. The compounds may be used for therapeutic purposes or research purposes. The ability of the inventive compounds to bind and/or stabilize the ryanodine receptor and prevent aberrant Ca²⁺ leakage makes these compounds useful in the treatment of cardiac disease, particularly cardiac arrhythmias associated with hear failure. In certain embodiments, the compound increase the affinity of calstabin2 for RyR2. The compounds stabilize the closed state of the RyR2 channel and prevent the leakage of Ca²⁺ from the sarcoplasmic reticulum that may cause cardiac arrhythmias. The compound are particularly useful in stabilizing complexes that include mutant calstabin2 or RyR2. The compound are also useful in stabilizing complexes that include phosphorylated or hyperphosphorylated RyR2. Phosphorylation of RyR2 results from beta-adrenergic stimulation. Calstabin2 typically dissociates from phosphorylated RyR2, thereby causing leakage of Ca²⁺ from the sarcoplasmic reticulum.

The invention provides a method of treating or preventing cardiac disease. The method involves the administration of a therapeutically effective amount of the compound or a pharmaceutically acceptable derivative thereof to a subject (including, but not limited to a human or animal) in need thereof. In certain embodiments, the subject suffers from a cardiac disease. In certain embodiments, the subject suffers from heart failure. In certain embodiments, the subject suffers from a cardiac arrhythmia. The arrhythmia may be an atrial and/or ventricular arrhythmia. In certain embodiments, the arrhythmia is atrial fibrillation. In certain embodiments, the arrhythmia is ventricular fibrillation. In certain embodiments, the arrhythmia is ventricular tachycardia. In certain embodiments, the arrhythmia is an exercise-induced cardiac arrhythmia. In certain embodiments, the cardiac disease is exercise-induced sudden cardiac death. In certain embodiments, the invention provides a method of preventing a fatal cardiac arrhythmia (e.g., a fatal ventricular arrhythmia).

The compounds and pharmaceutical compositions of the present invention may be used in treating or preventing any disease or conditions including cardiac diseases (e.g., arrhythmias). The compounds and pharmaceutical compositions may be administered to animals, preferably mammals (e.g., domesticated animals, cats, dogs, mice, rats), and more preferably humans. Any method of administration may be used to deliver the compound of pharmaceutical compositions to the animal. In certain embodiments, the compound or pharmaceutical composition is administered orally. In other embodiments, the compound or pharmaceutical composition is administered parenterally. In other embodiments, the compound or pharmaceutical composition is administered intravenously.

This invention also provides a pharmaceutical preparation comprising at least one of the compounds as described above and herein, or a pharmaceutically acceptable derivative thereof, which compounds stabilize the ryanodine (e.g., RyR2) receptor. In certain embodiments, the compound stabilizes the complex of RyR2 and calstabin2. In certain embodiments, the compound stabilizes the channel in the closed state.

As discussed above, the present invention provides novel compounds having cardioprotective activity, and thus the inventive compounds are useful for the treatment of cardiac arrhythmias. Accordingly, in another aspect of the present invention, pharmaceutical compositions are provided, wherein these compositions comprise any one of the compounds as described herein, and optionally comprise a pharmaceutically acceptable carrier. In certain embodiments, these compositions optionally further comprise one or more additional therapeutic agents, e.g., other cardiac medications. In other embodiments, these compositions further comprise a beta-blocker, an angiotensin-converting enzyme (ACE) inhibitor, calcium channel blocker, diuretic, vasodilator, inotropic agent, or natriuretic. In certain embodiments, the compositions includes an inventive compound and a beta-blocker. In certain embodiments, the beta-blocker is a beta1-selective agent. In certain embodiments, the beta-blocker is acebutolol, atenolol, betaxolol, bisoprolol, carvediol, celiprolol, esmolol, labetalol, metoprolol, or nebivolol.

In yet another aspect, according to the methods of treatment of the present invention, the affinity of calstabin2 for RyR2 is increased with an inventive compound or composition, as described herein. Thus, in still another aspect of the invention, a method for the treatment of a cardiac disease is provided comprising administering a therapeutically effective amount of an inventive compound, or a pharmaceutical composition comprising an inventive compound to a subject in need thereof, in such amounts and for such time as is necessary to achieve the desired result. In certain embodiments of the present invention a “therapeutically effective amount” of the inventive compound or pharmaceutical composition is that amount effective for stabilizing the closed form of the RyR2 channel. The compounds and compositions, according to the method of the present invention, may be administered using any amount and any route of administration effective for stabilizing the closed form of the RyR2 channel. The exact amount required will vary from subject to subject, depending on the species, age, and general condition of the subject, the severity of the disease, the particular compound, its mode of administration, its mode of activity, and the like. The compounds of the invention are preferably formulated in dosage unit form for ease of administration and uniformity of dosage. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. In certain embodiments, the therapeutically effective amount is the amount suffient to achieve plasma levels ranging from about 100 ng/ml to about 1000 ng/ml. In certain embodiments, the therapeutically effective amount is the amount suffient to achieve plasma levels ranging from about 300 ng/ml to about 1000 ng/ml. In certain embodiments, the therapeutically effective amount is the amount suffient to achieve plasma levels ranging from about 100 ng/ml to about 500 ng/ml. In certain embodiments, the therapeutically effective amount is the amount suffient to achieve plasma levels ranging from about 500 ng/ml to about 1000 ng/ml.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like. In certain embodiments, the compounds of the invention may be administered orally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect. The desired dosage may be delivered three times a day, two times a day, once a day, every other day, every third day, every week, or every two weeks. In certain embodiments, the desired dosage may be delivered using multiple administrations (e.g., two, three, four, five, six, seven, eight, nine, ten, eleven, twelve, thirteen, fourteen, or more administrations).

It will also be appreciated that certain of the compounds of the present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof, e.g., a prodrug.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19, 1977; incorporated herein by reference. The salts can be prepared in situ during the final isolation and purification of the compounds of the invention, or separately by reacting the free base functionality with a suitable organic or inorganic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

Additionally, as used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates. In certain embodiments, the esters are cleaved by enzymes such as esterases.

Furthermore, the term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

Liquid dosage forms for oral and parenteral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In certain embodiments for parenteral administration, the compounds of the invention are mixed with solubilizing agents such as Cremophor, alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

In still another aspect, the present invention also provides a pharmaceutical pack or kit comprising one or more containers filled with one or more of the ingredients of the pharmaceutical compositions of the invention, and in certain embodiments, includes an additional approved therapeutic agent for use as a combination therapy. Optionally associated with such container(s) can be a notice in the form prescribed by a governmental agency regulating the manufacture, use or sale of pharmaceutical products, which notice reflects approval by the agency of manufacture, use or sale for human administration.

### Screening of Inventive Compounds

The compounds described herein may be screened for any biological activity. In certain embodiments, the compounds are screened using known assays in the art. In certain embodiments, the compounds are screened for their ability to increase the affinity of calstabin2 for RyR2. See published U.S. patent application, US2005/0187386, published Aug. 25, 2005, which is incorporated herein by reference. In certain embodiments, the compounds are screened for their ability to stabilize the RyR2 channel in the closed state. For example, assays may be used to determined the concentration of the compound necessary to increase the binding of calstabin 2 to RyR2 by at least 10%, at least 25%, at least 50%, at least 75%, at least 90%, or at least 100% or more. Compounds with such activity may be useful for treating cardiac diseases such as ventricular arrhythmias, atrial arrhythmias, congestive heart failure, sudden death, or exercise-induced sudden death. In certain other embodiments, the compounds are tested for their ability to prevent leakage of Ca²⁺ through the RyR2 channel. In yet other embodiments, the compounds are tested for their ability to prevent arrhythmias in a non-human animal model such as those described in Wehrens et al., Science 304:292-296, 9 Apr. 2004, and published U.S. patent application, US 2005/0187386, published Aug. 25, 2005; each of which is incorporated herein by reference.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Synthetic Studies on JTV-519 and Its Analogues

The conformation of JTV-519 was studied in order to design analogues that would mimic a particular conformation found under in vivo conditions. It was assumed that JTV-519 probably existed as its protonated form in the body. Disappointingly, all of the attempts to obtain a single crystal of derivatives of JTV-519 failed. In the end, the amide side chain was replaced with a p-nitrobenzoyl group, and the X-ray structure of compound 2 was successfully obtained. With the X-ray structure in hand, various analogues of JTV-519 were designed, for example, adding methyl group substitutents on the seven-member ring. In addition, the oxygen analogs and simplified analogues were designed.

Compound 3 is a simplified analogue of JTV-519. It was assumed that compound 3 would adopt a similar conformation as compound 2. The advantage of compound 3 compared to JTV-519 is that it is much easier to synthesize. As shown in Scheme 1 below, the synthesis of 3 involves only four chemical transformations with very good yield for each step.

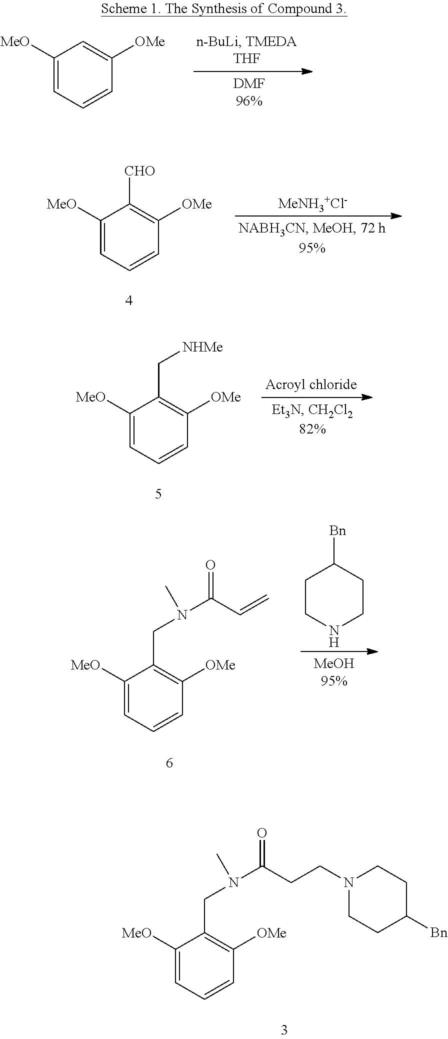

A challenge in this project was to develop an efficient route to access the seven-membered 1,4-benzothiazepine heterocycle, which could be further applied to the synthesis of analogues of JTV-519. Our initial synthetic route was based on published methodology. In the transformation of amine to the disulfide 7, it was found that the replacement of sodium disulfide with potassium ethyl xanthogenate substantially improved the yield of the reaction. However, we could not find high yielding conditions to efficiently convert compound 8 to 9. A number of bases (Cs₂CO₃, Na₂CO₃, K₂CO₃, Et₃N, DBU), different solvent systems (THF—MeOH—H₂O, THF—H₂O, MeOH—H₂O), and different reducing agents (PMe₃ and NaBH₄) were screened. The best yield that was obtained for this step was 42%. The whole sequence requires four steps and the overall yield is approximately 20%.

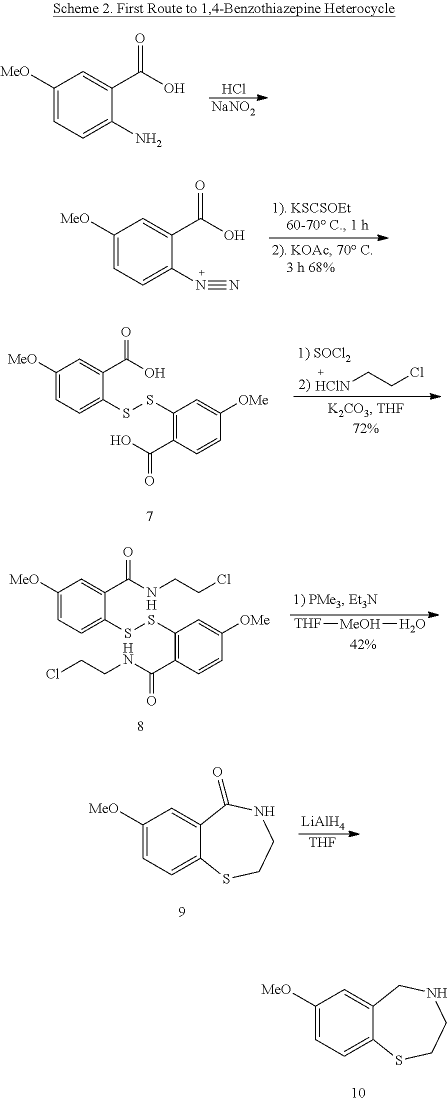

Although we could access a reasonable amount of the intermediate 9 using the original route, it is obviously not particularly efficient. Thus, we decided to explore an alternative approach. One attractive solution to the synthesis of 9 is to use a Schmidt rearrangement of known compound 12.³ However, very few examples of this kind of Schmidt rearrangement are described in the literature and most of the published procedures only delivered the product in low yield.⁴ To access ketone 12, commercially available 4-methoxybenzenethiol was first reacted with 3-bromopropyl acid to give acid 11 (Scheme 3). For the cyclization step, it was found the yield was significantly improved when a two stage one-pot procedure⁵ was used compared with the original literature procedure (polyphosphoric acid, ˜60%).

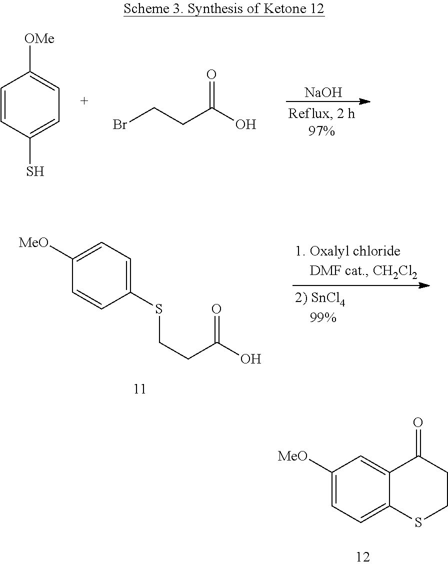

With compound 12 in hand, the Schmidt rearrangement step was explored. We first tried the reaction in DCM using 2 eq TFA and 1.5 eq TMSN₃. Disappointingly, only starting material was recovered after 2 days. TFA is one of the most popular solvent in Schmidt rearrangements. Compared to other popular solvents such as conc. HCl, its ability to dissolve the organic starting material makes the reaction easier to follow. The low acidity and no-oxidant property also render it very attractive. Disappointingly, the reaction of ketone 12 with both NaN₃ and TMSN₃ only provide the desired alkyl migrated compound in low yield, along with the aryl migrated product, substantial amounts of two teatrazoles, and a number of other side products (Scheme 4).

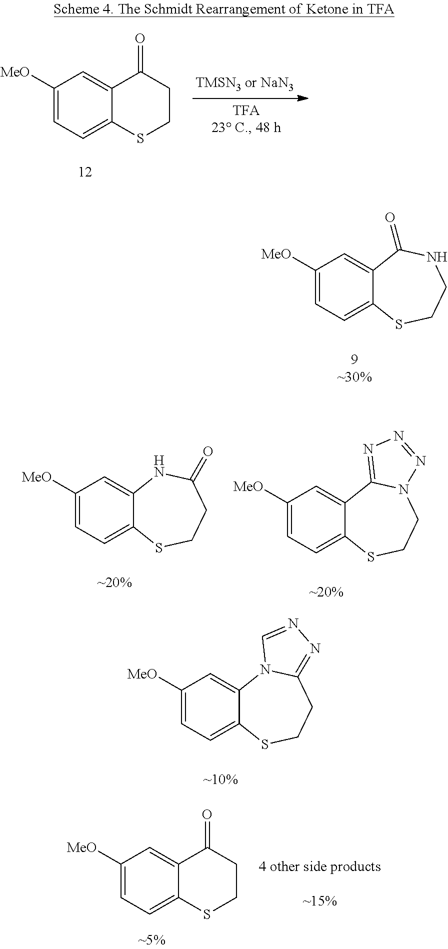

It was well documented that the Schmidt rearrangement of aromatic ketones in TFA generated a substantial amount of tetrazole as the major side products. However, no solution of this problem has been reported. We decided to explore conditions to minimize the formation of the tetrazoles; thus, making TFA a more useful solvent for this kind of transformation. The generally accepted mechanism of the Schmidt rearrangement involves formation of the iminodiazonium ion A (Scheme 5), followed by the migration of the anti-substituent to give intermediate B. Intermediate B could either react with water to give the amide C or react with excess HN₃ to give tetrazole side product D (Scheme 5). We reasoned that addition of suitable amount of water would favor the formation of amide. As a result, the amount of tetrazole would be reduced. However, large amount of water might also make the reaction very slow since it will convert the intermediate A back to the ketone.

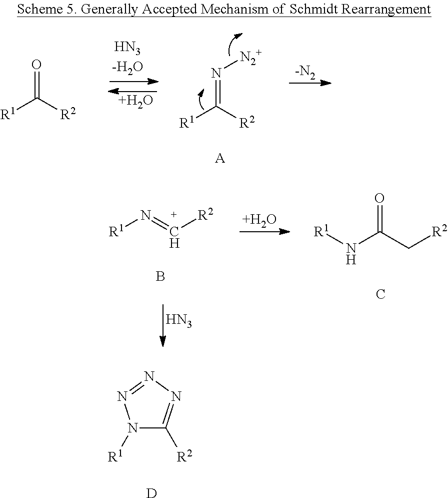

It was found that carrying out the reaction in TFA-H₂O (9:1) gave us satisfactory results (Scheme 6). The desired product was obtained in 55% yield (bsm) and the amount of tetrazoles were reduced to about 5%. Another unexpected benefit of using the new solvent system was that the ratio of desired alkyl-migration product and aryl-migration product was improved from 3:2 to 2:1. The reaction indeed slowed down (68 h, 90% conversion) as expected. Further increase of the amount of water in the solvent system made the reaction very slow. The reaction carried out in TFA-H₂O (3:1) only gave 25% conversion after 2 days. We also tested other common solvent system, including conc. HCl (˜40%) and H₂SO₄ in DCM (low conversion, low yield). The whole new sequence for the synthesis of 9 requires only three steps in an overall yield of greater than 50%.

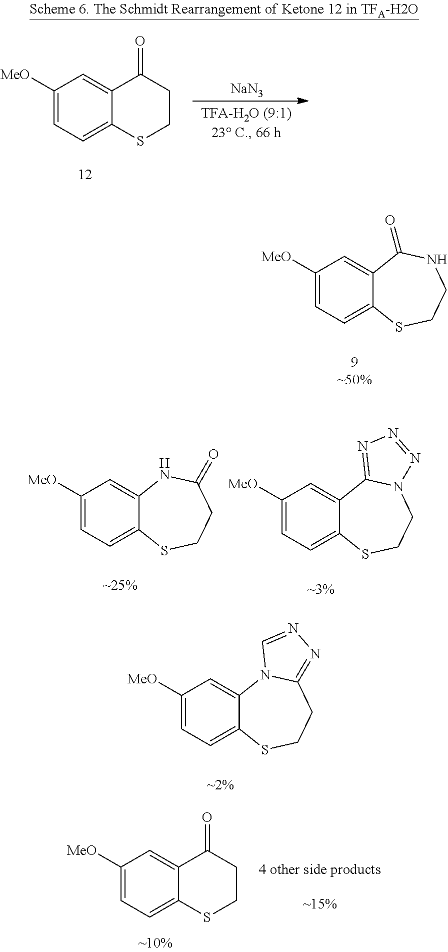

After the development of a more efficient route to the 1,4-benzothiazepine ring, we turned our attention to the preparation of analogs of JTV-519. We were very interested in the connections between the conformations of the seven-member ring and the biological activity of new analogues. Thus, we decided to make a number of derivatives of JTV-519 with methyl group on different positions of the ring. First, we prepared compound 19 (Scheme 7), with a methyl group at 2 position. Methyl (R)-3-hydroxybutyrate was converted into sulfide 13 in two steps using a closely related literature procedure.⁶ Although the substitution step in benzene was slow, the reaction gave the desired product in good yield and more importantly excellent enantioselectivity. To accelerate this step, we investigated using acetonitrile as the solvent. The reaction indeed proceeded faster; however, the product was only obtained in low enantioselectivity (<40% ee). Sulfide 13 was hydrolyzed by refluxing in conc. HCl and the resulting acid was converted to cyclic ketone 15 through a 2 stage one-pot procedure as in the synthesis of 12. The Schmidt rearrangement of 15 in TFA-H₂O with 1.5 eq NaN₃ gave the corresponding amide 16 in 58% yield. HPLC analysis showed the enantiomeric excess of 16 was about 95%. Amide 16 was then reduced to amine 17 with LiAlH₄, and 17 was transformed to 18 under standard conditions. Finally, compound 19 was synthesized by the Micheal addition of 4-benzylpiperidine to 18. In addition, the enantiomer of 19 was prepared in the same way, starting from methyl (S)-3-hydroxybutyrate.

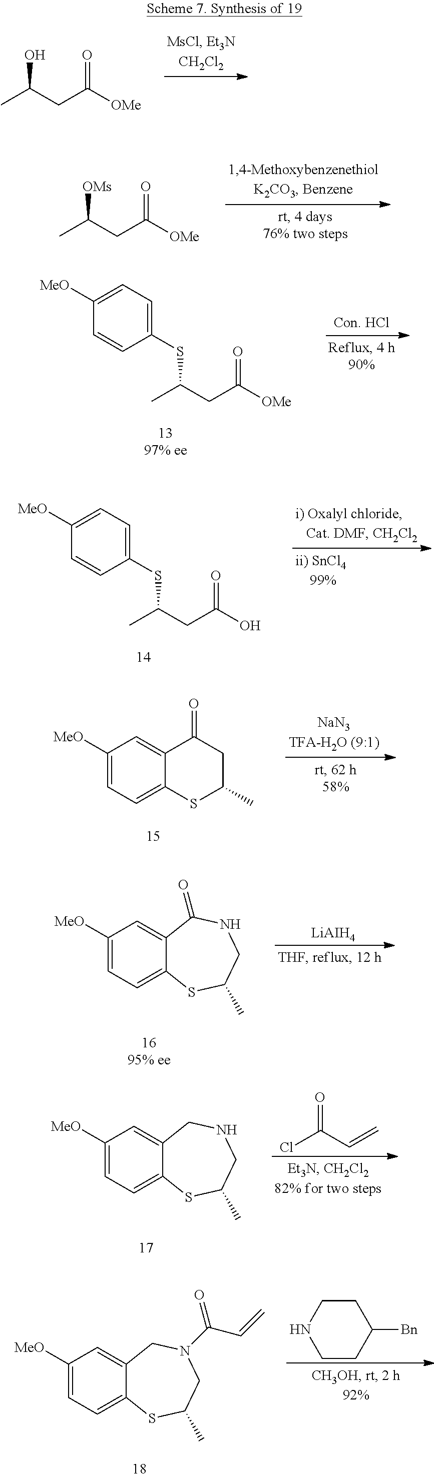

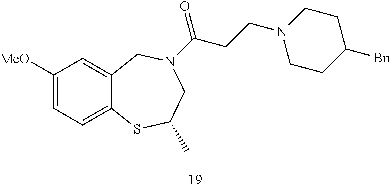

To synthesize 3-methyl-1,4-benzothiazepine, we initially tried to introduce a methyl group at position 3 of ketone 12 by alkylation. However, this reaction suffered elimination, and the product was only obtained in very low yield. As a result, an alternative strategy was employed (Scheme 8). 4-Methoxylthiophenol was reacted with methyl methacylate using a catalytic amount of n-BuLi to give the Michael product 20 in 98% yield. Compound 20 was then converted to compound 25 following the same procedure used in the synthesis of 18. At this stage, the two enantiomers of 18 were separated by preparative chiral HPLC and further transformed to compound 26 using a Micheal addition reaction.

The synthesis of 2,3-dimethyl-1,4-benzothiazepine was prepared via a similar route as the preparation of compound 26 (Scheme 8). Michael addition of 4-methoxylthiophenol to methyl tiglate yielded compound 27 as a mixture of diasteromers (erythro/threo 6:1).⁷Compound 27 was then converted to cis ketone 29 and trans ketone 30 (29/30 6:1) in two steps. The mixture of 29 and 30 was then subjected to the modified Schmidt rearrangement conditions, providing amide 31 as the major product with a small amount of trans isomer 32 which could not be removed by silica column chromatography. It is interesting to note that ketones 29 and 30 equilibrate in both acidic and basic media, slightly favoring the cis ketone 29. Pure amide 31 was obtained through recrystallization and transformed to 33 through a reduction/amide coupling two step sequence. At this stage, enantiomers of amide 33 could be separated by Chiralpak OD column and further transformed to compound 34 through a Micheal addition.

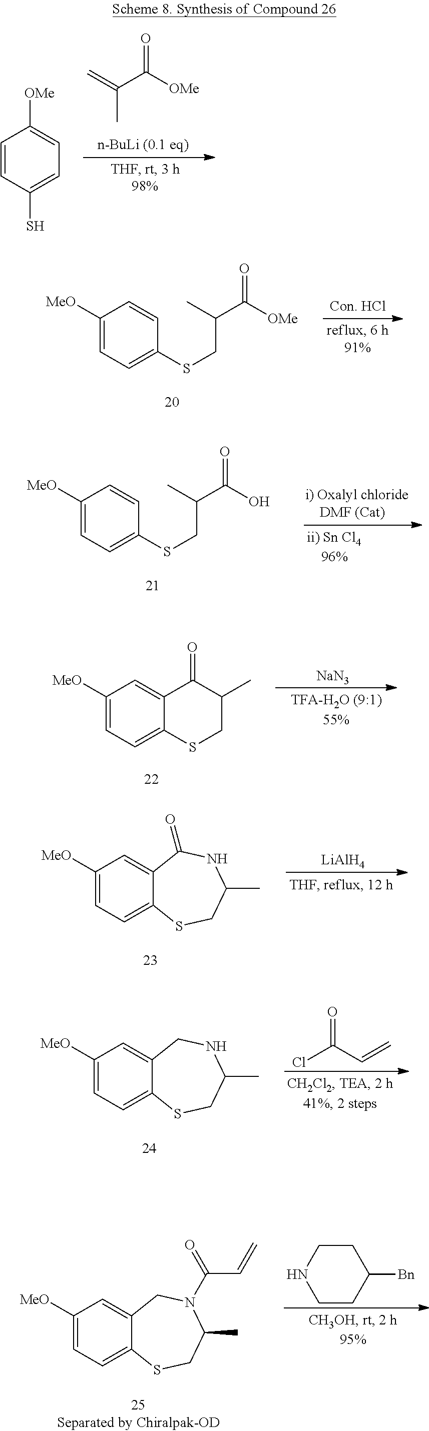

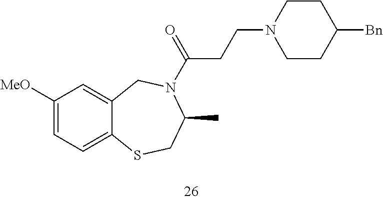

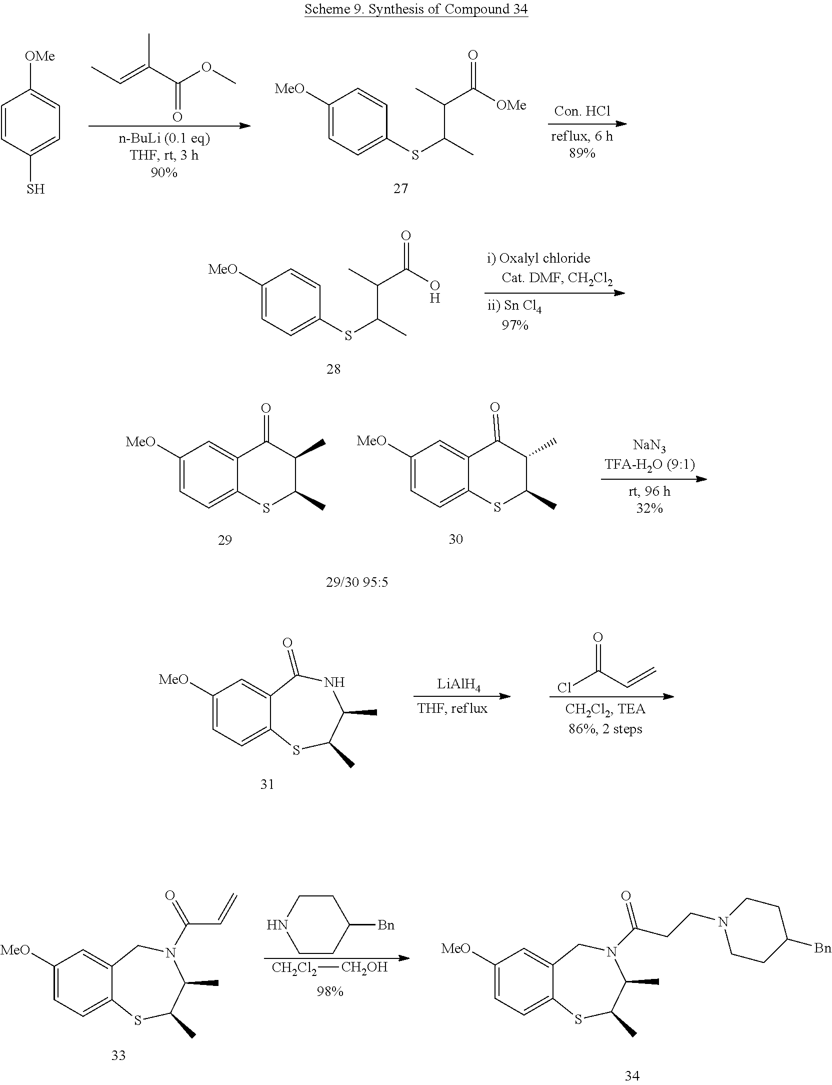

To make compound 32, we started with angelic acid methyl ester. Michael addition of 4-methoxylthiophenol to angelic acid methyl ester delivered compound 27 as a mixture of diasteromers (erythro/threo 1:2), which was then transformed into ketones 29 and 30 (Scheme 10). Surprisingly, it was found that the Schmidt rearrangement of ketone 30 is extremely slow compared to that of ketone 29. In addition, since ketones 29 and 30 equilibrate under Schmidt conditions. The final major product of the Schmidt rearrangement of a mixture of 29 and 30 (29/30 1:2) is compound 31 (31/32, 9:1).

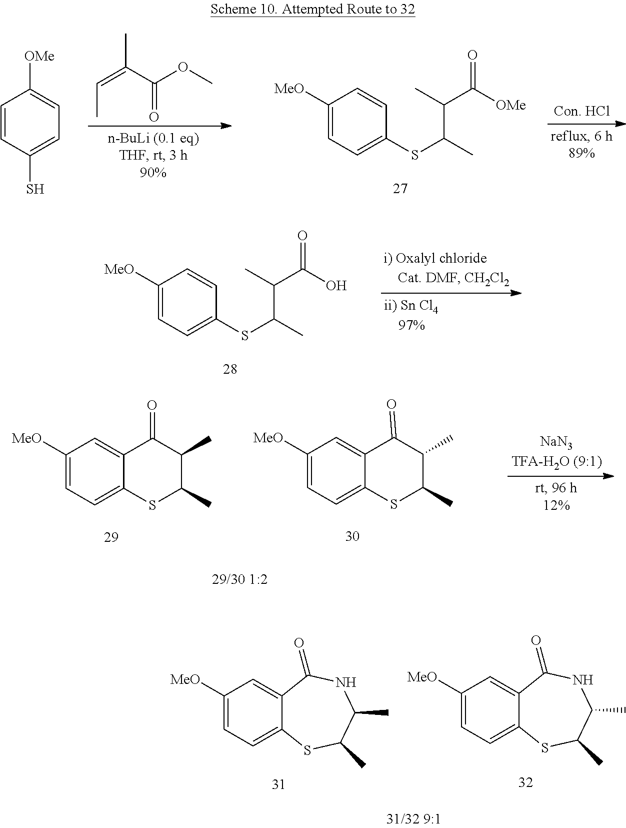

Replacing the sulfur atom with oxygen in JTV-519 might improve the metabolic stability significantly and at the same time preserve the bioactivity. The synthesis of 46, like the syntheses of other JTV-519 derivatives, again used the Schmidt rearrangement to construct the seven-membered heterocycle. Dithiane 39 was made from commercially available 2-hydroxy-6-methoxybenzaldehyde under standard conditions (Scheme 11). Treatment of 39 with 2 equiv. of n-BuLi and (+)-(R)- and (−)-(S)-propylene oxide gave the corresponding alcohol 40.¹⁰ Mitsunobu cyclization of diol 40 afford 41, which was further deprotected using Hg(ClO₄)₂ to deliver ketone 42. The Schmidt rearrangement of 42 proved to be a much cleaner reaction than that of its sulfur counterparts. The desired lactam 43 was obtained in 86% yield with 2.5 equiv. NaN₃. Lactam 43 was then converted to final compound 46 in a similar way to other JTV-519 analogues.

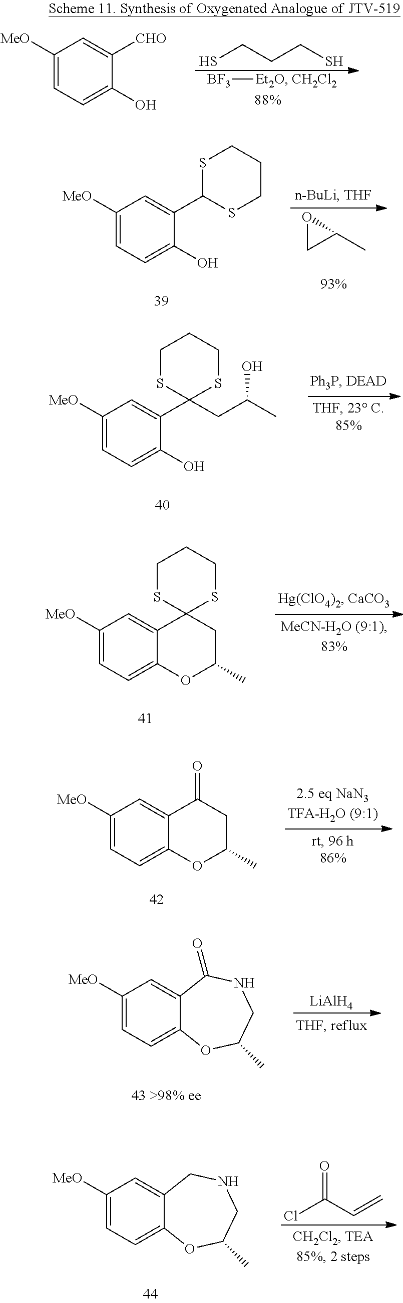

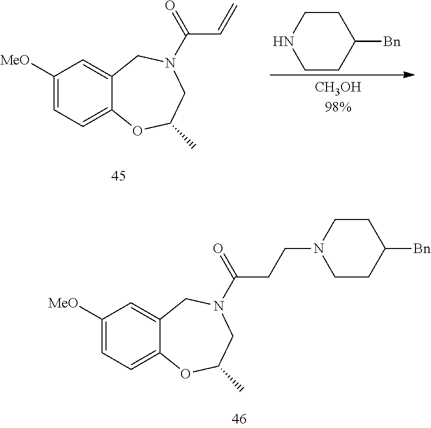

Finally, we synthesized two analogues with trisubtituted double bonds instead of an amide functional group (Scheme 12). The synthesis was initiated by the transformation of 3-butyn-1-ol into compound 47 in three steps. Sulfoxide 47 was subsequently converted to benzothiepinone 48 through gold (I)-catalyzed rearrangement developed by Toste group. Wittig olefination of 48 provided the desired compound 49 as an inseparable Z/E mixture (1:1). The alcohol in 49 was then converted to the mesylate, and the crude product was reacted with 4-benzylpiperidine without purification to produce 50. Subsequently, 50 was treated with HBr (generated from TMBr and water) to give the corresponding salt. Finally, the Z and E isomers of the salt were separated by recrystallization.

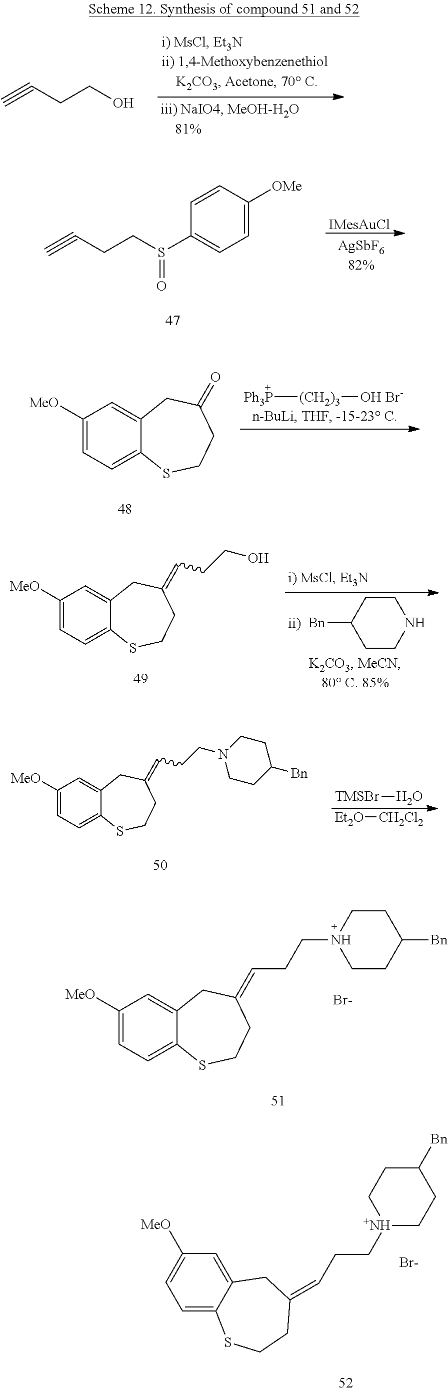

### Experimentals

Unless stated otherwise, reactions were performed in flame-dried glassware under a positive pressure of nitrogen using freshly distilled dry solvents. Thin-layer chromatography (TLC) was performed using E. Merck silica gel 60 F₂₅₄ precoated plates (0.25 mm) Flash chromatography was performed using Baker silica gel (40 μm particle size). NMR spectra were recorded on Varian Innova-500, or Mercury-400 instruments and calibrated using residual undeuterated solvent as an internal reference. IR spectra were recorded on Avatar 360 FT-IR spectrometer. Low-resolution and high-resolution mass spectral analyses were performed at the Harvard University Mass Spectrometry Center. Analytical high performance liquid chromatography (HPLC) was performed on Isco 2350 Series or Waters 626 HPLC using the indicated chiral column. Commercial grade reagents and solvents were used without further purification except as indicated below. Dichloromethane and MeCN were distilled from calcium hydride. Toluene, DME and THF were distilled from sodium.

### 2,6-Dimethoxybenzaldehyde (4)

A 250 mL RBF was charged with 1,3-dimethoxybenzene (5.4 g, 39 mmol), TMEDA (5.15 g, 44.3 mmol), and THF (60 mL). At 0° C., 2.5 M (18.7 mL, 46.85 mmol) was added. The solution was stirred at 0-5° C. for 30 mins and DMF (4.25 g, 58 mmol) was added slowly to maintain the temperature below 10° C. The solution was stirred or 1 h and quenched with 4N HCl. The solution was extracted with ethyl acetate, dried with sodium sulfate, and the solvent was removed under vacuum to give the title compound 4 in 73% yields (4.74 g).

### 2-(Methylaminomethyl)-1, 3-dimethoxybenzene (5)

A 100 mL RBF was charged with 2,6-Dimethoxybenzaldehyde (1.66 g, 10 mmol), methylamine hydrochloride (3.38 g, 50 mmol), sodium cyanoborohydride (0.76 g, 12 mmol) and methanol (50 mL). The reaction was stirred for 48 h at rt under nitrogen. After this period, drops of concentrated HCl were added to the reaction until the white cloudy gas stopped evolving. The reaction mixture was then concentrated in vaccu and then 30 mL of water added. The resulting mixture was extracted five times using 50 mL ethyl acetate each time. The organic layer was dried with sodium sulfate and concentrated to give the title compound 5 in 95% yields (1.72 g). ¹H-NMR (400 MHZ, CDCl₃): 2.40 (3H, s), 3.82 (3H, s), 6.53 (2H, d, J=8.6 Hz), 7.18 (1H, dd, J=8.6, 8.6 Hz).

### Compound 6

To a dichloromethane (3 mL) solution of 2-(Methylaminomethyl)-1, 3-dimethoxybenzene (1.38 g, 7.6 mmol) and Et₃N (2.32 g, 23 mmol) was added dropwise Acryloyl chloride (1.27 g, 11.4 mmol) at 0° C. After 1 h, the reaction mixture was warmed up to rt, stirred for another hour and then quenched with water. The reaction mixture was extracted with DCM, washed with 1N HCl, Sat. NaHCO₃ and brine, dried with anhydrous Na₂SO₄, and concentrated in vaccu to give the crude product. The crude product was purified by column chromatography (hexanes/ethyl acetate 1:3) to give the pure product in 82% yield (1.46 g) as a mixture of rotamers. The data of the major rotamer is as fellow. ¹H-NMR (400 MHZ, CDCl₃): 2.81 (3H, s), 3.79 (6H, s), 4.60 (2H, s), 5.63 (1H, d, J=1.8, 10.5 Hz), 6.29 (1H, dd, J=1.8, 17.0 Hz), 6.55 (2H, d, J=8.7 Hz), 7.06 (1H, dd, J=10.5, 17.0 Hz), 7.23 (1H, dd, J=8.7, 8.7 Hz).

### Compound 3

A mixture of compound 6 (0.47 g, 2 mmol) and 4-benzylpiperidine (0.53 g, 3 mmol) in DCM-MeOH (5 mL, v/v 1:1) was stirred at rt for 2 hours. The resulting mixture was then concentrated in vacuo to give the crude product. The crude product was purified by column chromatography (DCM/MOH 97:3) to give the pure product in 95% yield (0.78 g) as a mixture of rotamers. The data of the major rotamer is as fellow. IR: 2973, 1635, 1595, 1475 cm⁻¹; ¹H-NMR (400 MHZ, CDCl₃): 1.27-1.39 (1H, m), 1.50-1.70 (4H, m), 1.93-2.03 (2H, m), 2.50-3.00 (11H, m) (3H, s), 3.79 (6H, s), 4.60 (2H, s), 5.63 (1H, d, J=6.9, 1.8 Hz), 6.29 (1H, dd, J=10.5, 1.8 Hz), 6.55 (2H, d, J=8.6 Hz), 7.23 (1H, dd, J=8.6, 8.6 Hz); ¹³C-NMR (400 MHZ, CDCl₃): 172.2, 171.4, 159.6, 159.2, 140.8, 140.8, 129.7, 129.3, 129.3, 128.4, 126.0, 112.9, 112.4, 103.8, 55.9, 55.8, 55.1, 54.7, 54.2, 54.1, 43.4, 41.7, 38.7, 38.1, 38.0, 33.5, 32.4, 32.4, 31.8, 31.7, 31.0; MS (AP+) C₂₅H₃₅N₂O₃ (MH+) 411.2.

### Compound 11

To a degassed aqueous solution (20 mL) of NaOH (1.6 g, 40 mmol), 4-methoxylbenzenethiol (2.8 g, 20 mmol) and 3-bromopropyl acid (3.06 g, 20 mmol) was added under nitrogen. The reaction mixture was then reflux for 2 hours. After cooled to rt, the resulting aqueous solution was washed with ethyl acetate (20 mL). The aqueous layer was acidified with concentrated HCl, extracted with ethyl acetate (6×20 mL). The combined organic layer was then washed with brine (10 mL), dried with sodium sulfate, filtrated, and concentrated to afford the title compound in 97% yield (4.1 g). ¹H-NMR (400 MHZ, CDCl₃): 2.60 (2H, t, J=7.6 Hz), 3.05 (2H, t, J=7.6 Hz), 3.80 (3H, s), 6.83 (2H, d, J=8.7 Hz), 7.38 (2H, d, J=8.7 Hz).

### Compound 12

To a DCM (5 mL) solution of acid 11 (1.06 g, 5 mmol), DMF (18.5 uL) was added oxalyl chloride (0.70 g, 5.5 mmol) at rt under nitrogen. After 1 h, the solution was cooled to −10° C., and SnCl₄ (2.5 mL 1M solution, 2.5 mmol) was added dropwise. The mixture was then stirred at 0° C., after 0.75 hour, water (3 mL) was added. The mixture was then separated and the organic extract was washed with sat. Na₂CO₃, water, and brine, dried, filtered, and concentrated to give the title compound in 99% yield (0.96 g). ¹H-NMR (400 2.8 Hz), 7.38 (1H, d, J=8.7 Hz), 7.65 (1H, d, J=2.8 Hz).

### 7-Methoxy-5-oxo-2,3,4,5-tetrahydro-1,4-benzothiazepine (9)

To a solution of 12 (0.20 g, 1.0 mmol) in TFA-H₂O (5 mL, v/v 9:1) was added NaN₃ (50 mg, 0.75 mmol). After being stirred at rt for 24 h under nitrogen, same amount of NaN₃ was added to the reaction mixture. After 42 h, the reaction mixture was then gently poured into a mixture of ice and solid K₂CO₃ and basified to PH˜10. The aqueous solution was extrated with DCM and the organic layer was washed with water, brine, dried, filtered, and concentrated. The title compound was isolated by column chromatography (DCM/EtOAc 1:1) in 51% yield (56% yield based on 10% recovered starting material). ¹H-NMR (400 MHZ, CDCl₃): 3.10 (2H, t, J=7.3 Hz), 3.36 (2H, dt, J=7.3, 7.3 Hz), 3.83 (3H, s), 6.91 (1H, brs), 6.93 (1H, dd, J=8.5, 2.9 Hz), 7.22 (1H, d, J=2.9 Hz), 7.42 (1H, d, J=8.5 Hz). MS (AP+) C₁₀H₁₁NO₂S (MH+) 210.0.

### Compound 13

To a solution of methyl (R)-3-hydroxybutyrate (1.18 g, 10 mmol) and triethyl amine (1.31 g, 13 mmol) in dry DCM (13 mL) was added methanesulphonylchloride (1.49 g, 13 mmol) at 0° C. and the reaction mixture stirred at 0° C. for 2 hours. Water was then added and the mixture was extracted with DCM. The combined organic layers was washed with 1N HCl, sat. NaHCO₃, brine, dried and concentrated in vacuo to give the product as oil which was used for the next step without purification.

To a stirred mixture of the above sulphone ester and anhydrous K₂CO₃ (2.76 g, 20 mmol) in benzene (12 mL) was added 4-methoxylbenzenethiol (1.68 g, 12 mmol) at rt and the resulting mixture stirred at rt for 96 h and then quenched with water. The organic layer was washed with aq. 5% NaOH, water, brine, dried with Na₂SO₄, and concentrated to dryness. The residue was chromatographed (hexanes/ether 9:1) over silica gel to give the title compound as an oil in 76% yield (1.82 g) for two steps. Chiral HPLC analysis (Chiralpack OD-H hexanes/isopropnaol, 98:2, 1.0 mL/min 254 nm, t_{R}(major)=8.10 min., t_{R}(minor)=6.98 min.) 97% ee. [a]²³_{D} 21.9 (c=0.9, MeOH); IR: 2954, 1735, 1592, 1493 cm⁻¹; ¹H-NMR (400 MHZ, CDCl₃): 1.27 (3H, d, J=7.2 Hz), 2.39 (1H, dd, J=8.4, 15.5 Hz), 2.58 (1H, dd, J=6.4, 15.5 Hz), 3.39-3.48 (1H, m), 3.66 (3H, s), 3.80 (3H, s), 6.85 (2H, d, J=8.8 Hz), 7.41 (2H, d, J=8.8 Hz); ¹³C-NMR (400 MHZ, CDCl₃): 172.1, 160.1, 136.6, 123.8, 114.7, 55.5, 41.8, 40.7, 21.0; MS (AP+) C₁₀H₁₁NO₂S (MH+) 210.0.

### Compound 14

A suspension of compound 13 (0.9 g,) and 12 N HCl was refluxed under nitrogen for 4 hours. After cooled to rt, the reaction mixture was poured into ice and basified by K₂CO₃ to PH˜10. The mixture was then washed with ethyl acetate and the aqueous layer was acidified to PH˜2 by conc. HCl. The resulting mixture was extracted by ethyl acetate for 5 times. The combined organic layer was washed with brine, dried over Na₂SO₄, concentrated in vacuo to give the desired product in 90% yield. [a]²³_{D} 27.5 (c=1.0, MeOH); IR: 2992, 2963, 1701, 1492, 1243 cm⁻¹; ¹H-NMR (400 MHZ, CDCl₃): 1.30 (3H, d, J=6.6 Hz), 2.43 (1H, dd, J=8.1, 15.7 Hz), 2.62 (1H, dd, J=6.2, 15.7 Hz), 3.38-3.46 (1H, m), 3.80 (3H, s), 6.85 (2H, d, J=8.8 Hz), 7.41 (2H, d, J=8.8 Hz); ¹³C-NMR (400 MHZ, CDCl₃): 177.8, 160.2, 136.8, 123.4, 114.8, 55.6, 41.7, 40.3, 21.0; MS (AP+)

### Compound 15

The same procedure as for compound 12.

[a]²³_{D}-142 (c=0.8, MeOH); IR: 1667, 1600 cm⁻¹; ¹H-NMR (400 MHZ, CDCl₃): 1.43 (3H, d, J=6.9 Hz), 2.72 (1H, dd, J=11.7, 16.7 Hz), 3.00 (1H, dd, J=2.9, 16.7 Hz), 3.56-3.66 (1H, m), 3.83 (3H, s), 7.02 (1H, dd, J=2.9, 8.4 Hz), 7.17 (1H, and, J=8.4 Hz), 7.61 (1H, d, J=2.9 Hz); ¹³C-NMR (400 MHZ, CDCl₃): 194.9, 157.6, 133.4, 131.4, 129.0, 122.7, 111.3, 55.8, 48.2, 36.9, 20.6; MS (AP+)

### Compound 16

The same procedure as compound 9.

[a]²³_{D}-33.5 (c=0.5, MeOH); IR: 3217, 2929, 1656, 1649, 1593 cm⁻¹; ¹H-NMR (400 MHZ, CDCl₃): 1.27 (3H, d, J=6.6 Hz), 2.88-2.95 (1H, m), 3.30-3.37 (1H, m), 3.49-3.55 (1H, m), 3.83 (3H, s), 6.55 (1H, brs), 6.93 (1H, dd, J=2.9, 8.3 Hz), 7.24 (1H, d, J=2.9 Hz), 7.61 (1H, d, J=8.3 Hz); ¹³C-NMR (500 MHZ, CDCl₃): 172.8, 160.6, 141.5, 136.2, 120.8, 117.8, 114.5, 55.8, 47.1, 47.0, 20.0; MS (AP+) C₁₀H₁₁NO₂S (MH+) 223.9. Chiral HPLC analysis (Chiralpack OD-H hexanes/isopropnaol, 85:15, 1.0 mL/min, 254 nm, t_{R}(major)=14.4 min., t_{R}(minor)=12.2 min.) 95% ee.

### Compound 17

[a]²³_{D} 20.4 (c=0.5, MeOH); ¹H-NMR (400 MHZ, CDCl₃): 1.16 (3H, d, J=6.8 Hz), 2.75-2.79 (1H, m), 3.05 (1H, dd, J=7.8, 14.2 Hz), 3.42 (1H, dd, J=2.3, 14.2 Hz), 3.78 (3H, s), 4.07 (2H, d, J=5.5, 16.1 Hz), 6.67 (1H, dd, J=2.7, 8.2 Hz), 6.76 (1H, d, J=2.7 Hz), 7.45 (1H, d, J=8.2 Hz); ¹³C-NMR (400 MHZ, CDCl₃): 159.6, 148.8, 135.0, 125.7, 115.1, 112.2, 60.4, 55.6, 55.2, 43.2, 18.9; MS (AP+).

### Compound 18

[a]²³_{D} 1.8 (c=0.3, MeOH); IR: 1646, 1595, 1419 cm⁻¹; ¹H-NMR (500 MHZ, CDCl₃): 1.15-1.25 (3H, m), 2.90-3.08 (2H, m), 3.81 (3H, s), 4.04-4.15 (m, 1H), 4.70 (2H, AB, J=5.5, 16.1 Hz), 5.63 (0.5H, dd, J=1.8, 10.5 Hz), 5.72 (0.5H, dd, J=1.8, 10.5 Hz), 6.26 (0.5H, dd, J=1.8, 17.0 Hz), 6.29 (0.5H, dd, J=1.8, 10.5 Hz), 6.47 (1H, dd, J=10.5, 17.0 Hz), 6.68-6.80 (1H, m), 6.85 (0.5H, d, J=2.5 Hz), 7.15 (0.5H, d, J=2.5 Hz), 7.40 (0.5H, d, J=8.7 Hz), 7.47 (0.5H, d, J=8.7 Hz); ¹³C-NMR (500 MHZ, CDCl₃): 166.7, 159.6, 159.8, 143.7, 135.6, 128.7, 128.4, 128.1, 127.6, 116.9, 116.3, 113.7, 112.4, 56.7, 55.7, 55.6, 54.1, 52.8, 41.5, 18.8, 18.6; MS (AP+) C₁₄H₁₇NO₂S (MH+) 264.0.

### Compound 19

[a]²³_{D} 2.3 (c=1, CHCl₃); IR: 2919, 1643, 1594, 1451 cm⁻¹¹H-NMR (500 MHZ, CDCl₃): 1.18-1.37 (5H, m), 1.46-1.56 (1H, m), 1.57-1.66 (2H, m), 1.88-1.98 (2H, m), 2.36-2.72 (7H, m), 2.80-3.02 (3H, m), 3.78 (3H, s), 3.96-4.08 (m, 1H), 4.66 (2H, AB, J=5.5, 16.1 Hz), 6.68 (0.5H, dd, J=2.5, 8.7 Hz), 6.73 (0.5H, dd, J=2.5, 8.7 Hz), 6.86 (0.5H, d, J=2.5 Hz), 7.08 (0.5H, d, J=2.5 Hz), 7.11-7.30 (5H, m), 7.39 (0.5H, d, J=8.7 Hz), 7.46 (0.5H, d, J=8.7 Hz); ¹³C-NMR (500 MHZ, CDCl₃): 172.1, 159.7, 159.5, 143.5, 140.8, 135.6, 129.3, 129.3, 128.4, 126.0, 116.8, 116.4, 113.6, 112.6, 77.5, 55.6, 55.6, 54.6, 54.4, 54.4, 54.3, 54.3, 54.3, 54.1, 52.3, 43.4, 41.6, 38.0, 32.3, 31.6, 18.7; MS (AP+) C₂₆H₃₄N₂O₂S (MH+) 438.9.

### Compound 20

4-Methoxylbenzenethiol (2.1 g, 15 mmol) was added at 0° C. to a solution of n-BuLi (0.40 mL 2.5 M solution in hexanes, 1.0 mmol) in THF (50 mL). To the resulting solution was added methyl methacrylate dropwise. After being stirred at room temperature for three hours, the mixture was diluted with ether. The solution was then washed with 5% NaOH solution, brine, dried over Na₂SO₄, and concentrated in vaccu to give the desired product in 99% yield (2.38 g). IR: 2951, 1733, 1493 cm⁻¹; ¹H-NMR (500 MHZ, CDCl₃): 1.24 (3H, d, J=6.8 Hz), 2.60-2.65 (1H, m), 2.81 (1H, dd, J=6.8, 13.2 Hz), 3.14 (1H, dd, J=7.3, 13.2 Hz), 3.67 (3H, s), 3.80 (3H, s), 6.85 (2H, d, J=8.8 Hz), 7.37 (2H, d, J=8.8 Hz); ¹³C-NMR (400 MHZ, CDCl₃): 175.7, 159.5, 134.2, 125.9, 114.8, 55.6, 52.0, 40.0, 39.7, 16.9 MS (AP+) C₁₀H₁₁NO₂S (MH+).

### Compound 21

IR: 2886, 1703, 1492 cm⁻¹; ¹H-NMR (500 MHZ, CDCl₃): 1.28 (3H, d, J=6.5 Hz), 2.39 (1H, dd, J=8.4, 15.5 Hz), 2.60-2.68 (1H, m), 2.82 (1H, dd, J=6.8, 13.7 Hz), 3.16 (1H, dd, J=7.3, 13.7 Hz), 3.80 (3H, s), 6.85 (2H, d, J=8.8 Hz), 7.39 (2H, d, J=8.8 Hz); ¹³C-NMR (400 MHZ, CDCl₃): 181.2, 159.6, 134.4, 125.6, 114.9, 55.6, 39.8, 39.4, 16.7; MS (AP+) C₁₀H₁₁NO₂S (MH+).

### Compound 22

IR: 1671, 1474, 1266 cm⁻¹; ¹H-NMR (500 MHZ, CDCl₃): 1.35 (3H, d, J=6.5 Hz), 2.86-2.96 (1H, m), 3.06-3.18 (2H, m), 3.82 (3H, s), 6.95 (1H, dd, J=2.6, 8.8 Hz), 7.16 (1H, d, J=8.8 Hz), 7.62 (1H, d, J=2.6 Hz); ¹³C-NMR (400 MHZ, CDCl₃): 196.8, 157.6, 133.4, 131.4, 128.8, 122.3, 111.9, 55.8, 42.6, 33.6, 15.3; MS (AP+) C₁₀H₁₁NO₂S (MH+).

### Compound 23

IR: 3182, 3063, 1653, 1264 cm⁻¹; ¹H-NMR (500 MHZ, CDCl₃): 1.34 (3H, d, J=6.5 Hz), 2.72 (1H, dd, J=11.9, 11.9 Hz), 3.25 (1H, dd, J=3.6, 11.9 Hz), 3.39-3.48 (1H, m), 3.83 (3H, s), 6.92 (1H, dd, J=3.0, 8.5 Hz), 7.22 (1H, d, J=3.0 Hz), 7.41 (1H, d, J=8.5 Hz); ¹³C-NMR (400 MHZ, CDCl₃); MS (AP+) C₁₀K₁NO₂S (MH+).

### Compound 24

¹H-NMR (500 MHZ, CDCl₃): 1.13 (3H, d, J=6.8 Hz), 2.37 (1H, dd, J=9.8, 14.2 Hz), 2.75 (1H, d, J=14.2 Hz), 3.22-3.28 (1H, m), 3.77 (3H, s), 3.95 (1H, d, J=14.3 Hz), 4.23 (1H, d, J=14.3 Hz), 6.67 (1H, dd, J=2.5, 8.3 Hz), 7.78 (1H, d, J=2.5 Hz), 7.44 (1H, d, J=8.3 Hz); ¹³C-NMR (500 MHZ, CDCl₃): 171.4, 160.6, 142.0, 135.7, 120.8, 117.8, 114.6, 55.8, 47.9, 43.8, 19.6; MS (AP+) C₁₀K₁NO₂S (MH+).

### Compound 25

[a]²³_{D} 54.8 (c=1.0, MeOH); IR: 2933, 1643, 1596, 1419 cm⁻¹; ¹H-NMR (500 MHZ, CDCl₃): 1.4 1(1.6H, d, J=6.8 Hz), 1.53 (1.4H, d, J=6.8 Hz), 2.81-2.98 (2H, m), 3.77 (3H, s), 4.40-4.43 (1H, m), 4.48-4.52 (0.5H, m), 4.72 (0.5H, d, J=16.5 Hz), 4.95 (0.5H, d, J=15.0 Hz), 5.27-5.35 (0.5H, m), 5.64 (0.5H, dd, J=1.5, 10.5 Hz), 5.69 (0.5H, dd, J=1.5, 10.5 Hz), 6.21 (0.5H, dd, J=1.5, 16.5 Hz), 6.28 (0.5H, dd, J=1.5, 16.5 Hz), 6.52 (0.5H, dd, J=10.5, 16.5 Hz), 6.63 (0.5H, dd, J=10.5, 16.5 Hz), 6.68 (0.5H, dd, J=2.5, 8.5 Hz), 6.71 (0.5H, dd, J=2.5, 8.5 Hz), 6.80 (0.5H, d, J=2.5 Hz), 7.10 (0.5H, d, J=2.5 Hz), 7.34 (1H, d, J=8.3 Hz), 7.38 (1H, d, J=8.3 Hz); ¹³C-NMR (500 MHZ, CDCl₃): 166.9, 166.4, 159.4, 159.0, 133.4, 132.8, 128.7, 128.6, 128.3, 127.7, 127.5, 16.8, 113.6, 112.4, 55.7, 55.6, 52.4, 48.4, 46.7, 44.7, 40.7, 39.3, 29.9, 17.5, 16.8; MS (AP+) C₁₀H₁₁NO₂S (MH+). Chiral HPLC separation (Chiralpack OD hexanes/isopropnaol, 9:1, 5.0 mL/min, 254 nm).

### Compound 26

[a]²³_{D}-1.8 (c=0.5, CHCl₃); IR 2914, 1636, 1596, 1410 cm⁻¹; ¹H-NMR (400 MHZ, CDCl₃): 1.18-1.62 (8H, m), 1.82-1.98 (2H, m), 2.39-2.92 (10H, m), 3.76 (3H, s), 4.26-4.28 (1.5H, m), 4.66 (0.5H, d, J=16.5 Hz), 4.86 (0.5H, J=14.6 Hz), 5.23-5.32 (0.5H, m), 6.65 (0.5H, dd, J=2.8, 8.4 Hz), 6.68 (0.5H, dd, J=2.8, 8.4 Hz), 6.78 (0.5H, d, J=2.8 Hz), 7.04 (0.5H, d, J=2.8 Hz), 7.10-7.20 (3H, m), 7.22-7.38 (3H, m). ¹³C-NMR (400 MHZ, CDCl₃): 172.2, 170.9, 159.3, 159.0, 140.8, 140.8, 133.2, 132.8, 129.3, 128.4, 127.3, 126.0, 116.7, 116.3, 113.5, 112.6, 77.6, 55.6, 55.6, 54.6, 54.6, 54.5, 54.3, 54.1, 53.9, 46.6, 44.3, 43.4, 40.5, 39.2, 38.0, 32.4, 32.3, 31.9, 31.8, 17.5, 16.8; MS (AP+) C₂₆H₃₄N₂O₂S (MH+) 438.7.

Compound erythro-27

IR: 2949, 1731, 1492, 1242 cm⁻¹; ¹H-NMR (500 MHZ, CDCl₃): 1.26 (3H, d, J=7.0 Hz), 1.32 (3H, d, J=7.0 Hz), 2.58 (1H, dq, J=7.0, 7.0 Hz), 3.25 (1H, dq, J=7.0, 7.0 Hz), 3.63 (3H, s), 3.81 (3H, s), 6.83 (2H, d, J=8.6 Hz), 7.41 (2H, d, J=8.6 Hz); ¹³C-NMR (500 MHZ, CDCl₃): 175.3, 159.9, 136.4, 124.5, 114.7, 55.5, 51.8, 48.1, 45.3, 20.0, 14.9; MS (AP+) C₁₀H₁₁NO₂S (MH+).

Compound threo-27

¹H-NMR (500 MHZ, CDCl₃): 1.20 (6H, d, J=7.0 Hz), 2.60 (1H, dq, J=7.0, 7.0 Hz), 3.42 (1H, dq, J=7.0, 7.0 Hz), 3.69 (3H, s), 3.81 (3H, s), 6.85 (2H, d, J=8.6 Hz), 7.43 (2H, d, J=8.6 Hz); ¹³C-NMR (500 MHZ, CDCl₃): 175.5, 159.9, 136.1, 124.5, 114.7, 55.5, 51.9, 46.5, 44.1, 17.1, 12.7; MS (AP+) C₁₀H₁₁NO₂S (MH+).

Compound erythro-28

IR: 2990, 2968, 1701, 1492, 1242 cm⁻¹; ¹H-NMR (400 MHZ, CDCl₃): 1.32 (3H, d, J=7.0 Hz), 1.34 (3H, d, J=7.0 Hz), 2.60 (1H, dq, J=7.0, 7.0 Hz), 3.28 (1H, dq, J=7.0, 7.0 Hz), 3.81 (3H, s), 6.85 (2H, d, J=8.5 Hz), 7.42 (2H, d, J=8.5 Hz); ¹³C-NMR (500 MHZ, CDCl₃): 181.3, 160.0, 136.5, 124.2, 114.7, 55.5, 47.7, 45.2, 19.9, 14.5; MS (AP+) C₁₀H₁₁NO₂S (MH+).

Compound threo-28

¹H-NMR (500 MHZ, CDCl₃): 1.22 (6H, d, J=7.0 Hz), 2.62 (1H, dq, J=6.8, 7.0 Hz), 3.45 (1H, dq, J=6.8, 7.0 Hz), 3.80 (3H, s), 6.85 (2H, d, J=8.7 Hz), 7.42 (2H, d, J=8.7 Hz); ¹³C-NMR (500 MHZ, CDCl₃): 181.7, 160.0, 136.1, 124.3, 114.8, 46.0, 43.9, 16.9, 12.2; MS (AP+) C₁₀H₁₁NO₂S (MH+).

Compound cis-29

IR: 2970, 1672, 1598, 1373 cm⁻¹; ¹H-NMR (500 MHZ, CDCl₃): 1.25 (3H, d, J=7.3 Hz), 1.35 (3H, d, J=7.3 Hz), 3.00 (1H, dq, J=3.2, 7.3 Hz), 3.57 (1H, dq, J=3.2, 7.3 Hz), 3.82 (3H, s), 7.10 (1H, dd, J=2.8, 8.8 Hz), 7.13 (1H, d, J=8.8 Hz), 7.60 (1H, d, J=2.8 Hz); ¹³C-NMR (500 MHZ, CDCl₃): 197.7, 157.4, 132.5, 130.8, 128.9, 122.6, 111.6, 55.7, 47.4, 41.0, 16.2, 11.2; MS (AP+) C₁₀K₁NO₂S (MH+).

Compound trans-30

IR: 2970, 1672, 1598, 1373 cm⁻¹; ¹H-NMR (300 MHZ, CDCl₃): 1.34 (3H, d, J=7.3 Hz), 1.45 (3H, d, J=7.3 Hz), 2.68 (1H, dq, J=7.3, 7.5 Hz), 3.26 (1H, dq, J=7.3, 7.5 Hz), 3.82 (3H, s), 7.10 (1H, dd, J=2.8, 8.8 Hz), 7.13 (1H, d, J=8.8 Hz), 7.56 (1H, d, J=2.8 Hz); MS (AP+) C₁₀H₁₁NO₂S (MH+).

### Compound 31

IR: 3190, 3066, 1652, 1560 cm⁻¹; ¹H-NMR (500 MHZ, CDCl₃): 1.21 (3H, d, J=6.6 Hz), 1.23 (3H, d, J=6.6 Hz), 3.49-3.58 (2H, m), 3.83 (3H, s), 6.92 (1H, dd, J=2.9, 8.5 Hz), 7.24 (1H, d, J=2.9 Hz), 7.38 (1H, d, J=8.5 Hz); ¹³C-NMR (500 MHZ, CDCl₃): 171.5, 160.6, 141.5, 135.3, 122.1, 117.8, 114.7, 55.8, 50.2, 49.6, 17.1, 13.1; MS (AP+) C₁₂H₁₅NO₂S (MH+) 237.7.

### Compound 33

¹H-NMR (500 MHZ, CDCl₃): 0.99 (3H, d, J=7.3 Hz), 1.09 (3H, d, J=7.3 Hz), 2.78-2.83 (1H, m), 3.46-3.52 (1H, m), 3.79 (3H, s), 3.96 (1H, d, J=15 Hz), 4.18 (1H, d, J=15 Hz), 6.69 (1H, dd, J=2.7, 8.3 Hz), 6.75 (1H, d, J=2.7 Hz), 7.40 (1H, d, J=8.3 Hz).

### Compound 33

[a]²³_{D}-7.2 (c=1.0, CHCl₃); IR: 2977, 1652, 1646, 1437, 1420 cm⁻¹; ¹H-NMR (500 MHZ, CDCl₃): 1.25 (1.5H, d, J=7.3 Hz), 1.26 (1.5H, d, J=7.3 Hz), 1.40 (1.5H, d, J=6.9 Hz), 1.49 (1.5H, d, J=6.9 Hz), 3.06 (0.5H, dq, J=2.8, 7.3 Hz), 3.13 (0.5H, dq, J=2.8, 7.3 Hz), 3.79 (3H, s), 4.29 (0.5H, dq, J=2.8, 6.9 Hz), 4.37 (0.5H, d, J=15.6 Hz), 4.38 (0.5H, d, J=13.7 Hz), 4.71 (0.5H, d, J=15.6 Hz), 4.88 (0.5H, d, J=13.7 Hz), 5.20 (0.5H, dq, J=2.8, 6.9 Hz), 5.61 (0.5H, dd, J=1.8, 10.5 Hz), 5.71 (0.5H, dd, J=1.8, 10.5 Hz), 6.16 (0.5H, dd, J=1.8, 16.9 Hz), 6.25 (0.5H, dd, J=1.8, 16.9 Hz), 6.50 (0.5H, dd, J=10.5, 16.9 Hz), 6.67 (0.5H, dd, J=2.8, 8.7 Hz), 6.69 (0.5H, dd, J=2.8, 8.7 Hz), 6.76 (0.5H, dd, J=10.5, 16.9 Hz), 6.83 (0.5H, d, J=2.8 Hz), 7.16 (0.5H, d, J=2.8 Hz), 7.39 (0.5H, d, J=8.7 Hz), 7.46 (0.5H, d, J=8.7 Hz); ¹³C-NMR (500 MHZ, CDCl₃): 166.7, 166.2, 159.7, 159.4, 144.4, 144.1, 134.6, 133.7, 128.8, 128.5, 128.4, 127.6, 127.5, 126.8, 116.9, 116.5, 113.6, 112.1, 77.5, 56.8, 55.7, 55.6, 51.5, 49.2, 47.7, 46.7, 44.6, 19.4, 19.1, 12.4, 11.6; MS (AP+) C₁₂H₁₅NO₂S (MH+). Chiral HPLC separation (Chiralpack OD hexanes/isopropnaol, 9:1, 5.0 mL/min, 254 nm).

### Compound 34

[a]²³_{D} 8.5 (c=1.5, CHCl₃); IR: 2914, 1636, 1595, 1411 cm⁻¹; ¹H-NMR (500 MHZ, CDCl₃): 1.21-1.38 (6H, m), 1.42-1.68 (5H, m), 1.86-2.02 (2H, m), 2.38-2.91 (8H, m), 3.03 (0.5H, dq, J=2.8, 7.3 Hz), 3.07 (0.5H, dq, J=2.8, 7.3 Hz), 3.78 (3H, s), 4.16 (0.5H, dq, J=2.8, 6.9 Hz), 4.28 (1H, d, J=15.6 Hz), 4.66 (0.5H, d, J=15.6 Hz), 4.81 (0.5H, d, J=13.7 Hz), 5.17 (0.5H, dq, J=2.8, 6.9 Hz), 6.65 (0.5H, dd, J=2.8, 8.7 Hz), 6.69 (0.5H, dd, J=2.8, 8.7 Hz), 6.85 (0.5H, d, J=2.8 Hz), 7.10-7.29 (5.5H, m), 7.38 (0.5H, d, J=8.7 Hz), 7.45 (0.5H, d, J=8.7 Hz); ¹³C-NMR (500 MHZ, CDCl₃): 171.8, 170.5, 159.6, 159.4, 144.6, 144.0, 140.8, 140.8, 134.6, 133.6, 129.3, 129.3, 128.4, 128.4, 127.6, 126.7, 126.0, 116.9, 116.5, 113.4, 112.5, 56.2, 55.6, 55.6, 54.7, 54.5, 54.4, 54.3, 54.0, 51.1, 49.1, 47.8, 46.6, 44.3, 43.4, 38.0, 37.9, 32.4, 32.3, 32.0, 19.5, 19.0, 12.3, 11.5; MS (AP+) C₁₂H₁₅NO₂S (MH+).

### Compound 39

Propane-1,3-dithiol (6.49 g, 60 mmol), BF₃-Et₂O (2.83 g, 20 mmol) and 2-hydroxy-6-methoxy-benzaldehyde (7.5 g, 50 mmol) was stirred in methylene chloride (100 mL) at rt for 16 h. The reaction mixture was then quenched with sat aqueous NaHCO₃ and the resulting mixture was extracted with methylene chloride. The combined organic layer was washed with brine, dried with Na₂SO₄, filtered and evaporated to give a white solid, which was recrystallized from ethyl acetate to give 39 in 85% (10.3 g). Mp 125-126° C.; ¹H-NMR (500 MHZ, CDCl₃): 1.79-1.98 (1H, m), 2.32-2.42 (1H, m), 2.91-2.95 (2H, m), 3.06-3.12 (2H, m), 3.78 (3H, s), 5.38 (1H, s), 5.86 (1H, s), 6.78-6.86 (3H, m); ¹³C-NMR (500 MHZ, CDCl₃): 153.8, 148.3, 124.6, 118.4, 116.1, 114.1, 6.0, 47.7, 31.9, 25.1.

### Compound 40

To a solution of 39 (1.94 g, 8.0 mmol) in THF (50 mL) was added n-BuLi (8.0 mL 2.5 M solution in hexanes, 20 mmol) at −15° C. After being stirred for 3 h, (+)-(R)-propylene oxide (0.93 g, 16 mmol) was added. After stirred for another 3 h, the reaction was quenched with sat. aqueous NH₄Cl. The resulting mixture was then extracted with ethyl acetate. The combined organic layer was then washed with brine, dried with Na₂SO₄, filtered, and evaporated in vacuo. The crude mixture was then purified by column chromatography (hexanes/ethyl acetate 2:1) to afford 40 in 87% yields (2.10 g). [a]²³_{D} 5.9 (c=0.9, MeOH); IR: 3213, 2971, 1653, 1480, 1043 cm⁻¹; ¹H-NMR (500 MHZ, CDCl₃): 1.11 (3H, d, J=6.4 Hz), 1.86-2.06 (3H, m), 2.17 (1H, dd, J=2.3, 14.7 Hz), 2.28 (1H, dd, J=8.7, 14.7 Hz), 2.70-2.77 (2H, m), 2.86-2.96 (2H, m), 3.79 (3H, s), 4.01-4.06 (1H, m), 6.83 (1H, dd, J=3.2, 8.7 Hz), 6.88 (1H, d, J=8.7 Hz), 7.52 (1H, d, J=3.2 Hz), 8.12 (1H, s); ¹³C-NMR (500 MHZ, CDCl₃): 153.6, 149.8, 124.9, 120.4, 117.3, 115.3, 64.9, 57.2, 56.0, 50.9, 28.3, 28.0, 24.6, 24.2.

### Compound 41

To a solution of Ph₃P (8.2 g, 31.2 mmol) in THF (50 mL) at rt was added DEAD (2.53 g, 14.5 mmol). After stirred for 1 h, the reaction mixture was added to 40 (2.36 g, 7.8 mmol) in THF (10 mL). After 1 h, the reaction was quenched with water and extracted with ether. The combined layers were then washed with brine, dried with Na₂SO₄, evaporated. The crude mixture was purified by column chromatography (hexanes/ethyl acetate 10:1) to deliver 41 in 85% yields (1.87 g). [a]²³_{D}-13.3 (c=0.5, MeOH); IR: 2904, 1614, 1489, 1214 cm⁻¹; ¹H-NMR (500 MHZ, CDCl₃): 1.43 (3H, d, J=6.4 Hz), 1.93-2.03 (1H, m), 2.15-2.25 (2H, m), 2.62-2.68 (1H, m), 2.78-2.83 (1H, m), 2.92 (1H, dd, J=2.3, 13.7 Hz), 3.08-3.22 (2H, m), 3.79 (3H, s), 4.32-4.39 (1H, m), 6.83 (1H, dd, J=3.2, 8.7 Hz), 6.72-6.80 (2H, m), 7.35 (1H, d, J=2.3 Hz); ¹³C-NMR (500 MHZ, CDCl₃): 153.7, 149.2, 124.2, 118.0, 117.3, 113.6, 69.7, 56.0, 48.9, 43.3, 28.2, 28.1, 24.9, 21.0.

### Compound 42

To a suspension of compound 41 (1.13 g, 4.0 mmol.) and CaCO₃ (0.6 g, 6.0 mmol) in THF—H₂O (18 mL, v/v 5:1) was added Hg(ClO₄)₂ (1.5 mL 4M water solution, 6.0 mmol) at 0° C. After stirred for 1 h, the mixture was filtered and extracted with ether. The combined layers were then washed with brine, dried with Na₂SO₄, evaporated. The crude mixture was purified by column chromatography (hexanes/ethyl acetate 10:1) to deliver 42 in 83% yields (0.64 g). [a]²³_{D}-77.2 (c=0.5, CHCl₃); IR: 2922, 1680, 1652, 1617, 1482, 1216 cm⁻¹; ¹H-NMR (500 MHZ, CDCl₃): 1.50 (3H, d, J=6.3 Hz), 2.65-2.68 (2H, m), 3.80 (3H, s), 4.52-4.58 (1H, m), 6.90 (1H, d, J=9.0 Hz), 7.09 (1H, dd, J=3.2, 9.0 Hz), 7.31 (1H, d, J=3.2 Hz); ¹³C-NMR (500 MHZ, CDCl₃): 192.8, 156.7, 154.1, 125.4, 120.8, 119.4, 107.4, 74.6, 56.0, 44.7, 21.2.

### Compound 43

The same procedure as other Schmidt rearrangement except using 2.5 equiv. NaN₃ and two times concentration. [a]²³_{D} 58.0 (c=1.0, MeOH); IR: 1661, 1482, 1260 cm⁻¹; ¹H-NMR (500 MHZ, CDCl₃): 1.32 (3H, d, J=6.9 Hz), 3.11 (1H, ddd, J=5.5, 6.9, 15.1 Hz), 3.35 (1H, ddd, J=4.1, 6.4, 15.1 Hz), 3.82 (3H, s), 4.51-4.57 (1H, m), 6.58 (1H, brs), 6.94 (1H, d, J=8.7 Hz), 6.99 (1H, dd, J=3.2, 8.7 Hz), 7.29 (1H, d, J=3.2 Hz); ¹³C-NMR (500 MHZ, CDCl₃): 172.2, 156.1, 147.6, 128.2, 124.0, 119.9, 113.5, 80.6, 55.9, 45.8, 18.3; MS (AP+) C₁₁H₁₃NO₃ (MH+) 207.8. Chiral HPLC analysis (Chiralpack OD-H hexanes/isopropnaol, 85:15, 1.0 mL/min, 254 nm, t_{R}(major)=11.3 min., t_{R}(minor)=14.1 min.) 98% ee.

### Compound 44

¹H-NMR (400 MHZ, CDCl₃): 1.29 (3H, d, J=6.4 Hz), 2.93 (1H, dd, J=9.2, 14.1 Hz), 3.18 (1H, d, J=14.1 Hz), 3.76 (3H, s), 3.75-3.80 (1H, m), 3.82 (1H, d, J=14.1 Hz), 4.00 (1H, d, J=14.1 Hz), 6.63-6.70 (2H, m), 6.95 (1H, d, J=8.7 Hz).

### Compound 45

[a]²³_{D} 38.6 (c=2.0, CHCl₃); IR: 2975, 1646, 1611, 1494 cm⁻¹; ¹H-NMR (500 MHZ, CDCl₃): 1.32 (1H, d, J=6.4 Hz), 1.35 (1H, d, J=6.4 Hz), 3.22 (0.6H, dd, J=9.0, 13.6 Hz), 3.53 (0.4H, dd, J=9.0, 13.6 Hz), 3.78 (3H, s), 3.88-3.99 (1H, m), 4.31 (0.6H, d, J=13.6 Hz), 4.47-4.60 (2H, m), 4.95 (0.4H, d, J=13.6 Hz), 5.66 (0.4H, dd, J=2.0, 10.2 Hz), 5.72 (0.6H, dd, J=2.0, 10.2 Hz), 6.28 (1H, dd, J=2.0, 16.3 Hz), 6.50 (0.4H, dd, J=10.2, 16.3 Hz), 6.65-6.75 (2.6H, m), 6.89-6.93 (0.4H, m), 6.97 (0.6H, d, J=8.8 Hz); ¹³C-NMR (500 MHZ, CDCl₃): 166.3, 165.8, 155.9, 155.7, 151.9, 151.5, 132.2, 128.6, 128.4, 127.9, 127.6, 123.0, 122.4, 115.3, 114.6, 114.0, 113.2, 78.3, 78.1, 56.7, 55.8, 55.8, 54.1, 51.4, 49.0, 19.6, 19.2.

### Compound 46

[a]²³_{D} 12.5 (c=1.0, CHCl₃); IR: 2914, 1645, 1495, 1216 cm⁻¹; ¹H-NMR (500 MHZ, CDCl₃): 1.21-1.33 (5H, m), 1.43-1.62 (3H, m), 1.85-1.98 (2H, m), 2.36-2.72 (6H, m), 2.80-2.91 (2H, m), 3.15 (0.6H, dd, J=9.0, 13.8 Hz), 3.43 (0.4H, dd, J=9.2, 15.0 Hz), 3.68-3.90 (4H, m), 4.16 (0.6H, d, J=13.7 Hz), 4.39-4.51 (2H, m), 4.92 (1H, d, J=14.0 Hz), 6.66-6.73 (1.5H, m), 6.86-6.97 (1.5H, m), 7.16-7.58 (m, 5H); ¹³C-NMR (500 MHZ, CDCl₃): 171.5, 170.7, 155.9, 155.7, 152.0, 151.8, 140.8, 140.8, 132.5, 132.1, 129.3, 128.4, 126.0, 123.1, 122.3, 115.2, 114.9, 114.0, 113.3, 78.5, 78.4, 77.5, 56.7, 55.9, 55.8, 54.5, 54.5, 54.4, 54.3, 54.2, 54.1, 53.9, 51.3, 48.6, 43.4, 38.0, 38.0, 32.3, 31.7, 31.3, 9.6; MS (AP+) C₂₆H₃₄N₂O₃ (MH+) 423.2.

### Compound 47

To a solution of 3-butyn-1-ol (1.40 g, 20 mmol) and triethyl amine (3.03 g, 30 mmol) in dry DCM (40 mL) was added methanesulfonyl chloride (2.98 g, 26 mmol) at 0° C. and the reaction mixture stirred at 0° C. for 2 hours. Water was then added and the mixture was extracted with DCM. The combined organic layers was washed with 1N HCl, sat. NaHCO₃, brine, dried and concentrated in vacuo to give the product as oil which was used for the next step without purification.

To a stirred mixture of the above sulfone ester and anhydr. K₂CO₃ (4.14 g, 30 mmol) in acetone (12 mL) was added 4-methoxylbenzenethiol (2.80 g, 20 mmol) at rt and the resulting mixture was refluxed until thiol is consumed (˜5 h). Cool to r. t., quench with brine, extract with ether, dry with Na₂SO₄, concentrated and purified with flash chromatography (hexanes/ether 9:1) to give the sulfide as an oil in 90% yield (3.85 g) for two steps.

To a solution of the sulfide (3.46 g, 18 mmol) in MeOH—H₂O (60 mL, v/v 9:1) was added NaIO₄ (4.62 g, 21.6 mmol) and stirred overnight. Upon completion, filter the reaction mixture through celite, concentrated under reduced pressure and purified via column chromatography (hexanes/ethyl acetate 5:1). The desired product was isolated in 95% yield (3.56 g). IR: 3050, 1594 1495, 1249, 1087, 1041 cm⁻¹; ¹H-NMR (500 MHZ, CDCl₃): 2.03 (1H, t, J=2.8 Hz), 2.35-2.45 (1H, m), 2.65-2.71 (1H, m), 2.90-2.99 (2H, m), 3.86 (3H, s), 7.04 (2H, d, J=8.8 Hz), 7.56 (2H, d, J=8.8 Hz); ¹³C-NMR (500 MHZ, CDCl₃): 162.3, 133.9, 126.1, 115.1, 81.0, 70.7, 55.7, 55.4, 12.3; MS (AP+) C₂₆H₃₄N₂O₃ (MH+).

### Compound 48

A 25 mL round bottom flask was charged with sulfoxide (0.583 g, 2.8 mmol), IMesAuCl (0.105 g, 0,196 mmol), AgSbF₆ (0.060 g, 0,168 mmol) and DCM (12 mL). The reaction was stirred for 1 h at rt. The reaction mixture was then concentrated in vaccu and the resulting residue was purified with flash chromatography (hexanes/EtOAc 4:1) to give the 48 as an oil in 88% yield (0.51 g). IR: 1706, 1594, 1264 cm⁻¹; ¹H-NMR (500 MHZ, CDCl₃): 2.82-2.85 (2H, m), 3.99-3.02 (2H, m), 3.80 (3H, s), 3.96 (3H, s), 6.74 (1H, dd, J=2.7, 8.7 Hz), 6.85 (1H, d, J=2.7 Hz), 7.45 (1H, d, J=8.7 Hz); ¹³C-NMR (500 MHZ, CDCl₃): 206.5, 160.4, 40.2, 135.6, 125.9, 116.2, 113.2, 55.6, 51.6, 45.4, 32.6; MS (AP+) C₂₆H₃₄N₂O₃ (MH+).

### Compound 49

n-BuLi (2.8 mL 2.5 M solution in hexanes, 7.0 mmol) was added dropwise to a stirred slurry of the phosphonium salt (1.55 g, 3.87 mmol) in THF (10 mL) at −15° C. The red-brown mixture was allowed to warm up to r.t. in 1 h and stirred at r.t. for 2 hours. 48 (0.324 g, 1.55 mmol) in THF (2 mL) was added to the suspension dropwise at −10° C. The resulting mixture was slowly warmed up to r.t. After being stirred overnight, the reaction was quenched with saturated aqueous NH₄Cl, extract with EtOAc, dry with Na₂SO₄, concentrated and purified with flash chromatography (hexanes/EtOAc 2:1) to give the 49 as an oil in 28% yield (0.109 g) along with 62% recovery of 48. IR: 3319, 1592, 1237, 1030 cm⁻¹; ¹H-NMR (500 MHZ, CDCl₃): 2.25 (2H, dt, J=6.5, 6.5 Hz), 2.44 (2H, dt, J=6.5, 6.5 Hz), 2.66-2.76 (8H, m), 3.58-3.62 (4H, m), 3.64-3.68 (4H, m), 3.79 (3H, s), 3.80 (3H, s), 5.23 (1H, t, J=6.5 Hz), 5.37 (1H, t, J=6.5 Hz), 6.64 (2H, dd, J=2.9, 8.3 Hz), 6.81 (1H, d, J=2.9 Hz), 6.85 (1H, d, J=2.9 Hz), 7.40 (1H, d, J=8.3 Hz), 7.43 (1H, d, J=8.3 Hz); ¹³C-NMR (500 MHZ, CDCl₃): 159.8, 159.7, 147.6, 146.4, 139.1, 139.0, 134.8, 134.5, 127.0, 126.7, 123.7, 123.5, 115.9, 115.2, 111.3, 111.0, 62.5, 55.6, 55.5, 46.5, 43.2, 39.0, 35.5, 34.6, 34.4, 31.5, 31.4; MS (AP+) C₂₆H₃₄N₂O₃ (MH+).

### Compound 50

To a solution of 49 (0.186 g, 0.743 mmol) and triethyl amine (0.19 g, 1.86 mmol) in dry DCM (2 mL) was added methanesulfonyl chloride (0.128 g, 1.12 mmol) at 0° C. and the reaction mixture stirred at 0° C. for 1 hour. Water was then added and the mixture was extracted with DCM. The combined organic layers was washed with 1N HCl, Sat. aq. NaHCO₃, brine, dried and concentrated in vacuo to give the product as oil which was used for the next step without purification.

To a stirred mixture of the above sulfone ester and anhydr. K₂CO₃ (0.21 g, 1.5 mmol) in acetonitrile (2 mL) was added 4-benzylpiperidine (0.196 g, 1.12 mmol) at rt and the resulting mixture was heated at 80° C. for 5 h and then quenched with water at r.t. The resulting mixture was extract with DCM, dry with Na₂SO₄, concentrated and purified with flash chromatography (DCM/MeOH 98:2) to give 50 as an oil in 85% yield (0.257 g) for two steps. IR: 2911, 1653, 1592, 1266 cm⁻¹; ¹H-NMR (500 MHZ, CDCl₃): 1.20-1.70 (10H, dm), 1.80-1.95 (4H, m), 2.16-2.40 (8H, m), 2.51-2.72 (12H, m), 2.83-2.96 (4H, m), 3.53 (2H, s), 3.62 (2H, s), 3.78 (3H, s), 3.78 (3H, s), 5.16 (1H, t, J=6.5 Hz), 5.31 (1H, t, J=6.5 Hz), 6.62 (1H, dd, J=2.7, 8.7 Hz), 6.63 (1H, dd, J=2.7, 8.7 Hz), 6.79 (1H, d, J=2.7 Hz), 6.84 (1H, d, J=2.7 Hz), 7.11-7.29 (5H, m), 7.39 (1H, d, J=8.7 Hz), 7.42 (1H, d, J=8.7 Hz); ¹³C-NMR (500 MHZ, CDCl₃): 159.8, 159.7, 147.9, 146.6, 140.9, 140.9, 137.1, 137.0, 134.8, 134.5, 129.4, 129.4, 128.4, 128.4, 127.1, 126.8, 126.1, 126.0, 125.4, 125.2, 116.0, 115.2, 111.2, 111.0, 58.9, 58.7, 55.6, 55.5, 54.1, 54.1, 46.5, 43.4, 43.2, 39.0, 38.1, 38.1, 35.5, 34.6, 34.5, 32.3, 25.8, 25.7; HRMS (ESI, m/z) calcd for C₂₆H₃₄NOS 408.2361. found 408.2350.

### Compound 51

To a solution of 50 (0.170 g, 0.417 mmol) and TMSBr (70.5 mg, 0.46 mmol) in DCM (2 mL) was added water (8.28 mg, 0.46 mmol) at r.t. and the reaction mixture stirred at r.t. for 15 minutes. The resulting mixture was then concentrated in vaccu to give the crude product as solid. The resulting solid mixture was then dissolved in DCM-EtOAc in a 25 mL flask. The solvent was slowly evaporated at r.t. to give 52 as white crystal. After filtration the mother solution was evaporated at reduced pressure to give 51 as light yellow solid. ¹H-NMR (500 MHZ, CDCl₃): 1.60-1.88 (3H, m), 2.00-2.18 (2H, m), 2.48-2.86 (12H, m), 3.51-3.56 (4H, m), 3.77 (3H, s), 5.23 (1H, t, J=7.0 Hz), 6.61 (1H, dd, J=2.9, 8.8 Hz), 6.77 (1H, d, J=2.9 Hz), 7.11-7.30 (5H, m), 7.38 (1H, d, J=8.8 Hz), 11.30 (1H, brs); ¹³C-NMR (500 MHZ, CDCl₃):159.9, 146.9, 140.9, 139.2, 134.7, 129.2, 128.8, 126.7, 120.8, 115.2, 111.4, 57.3, 55.6, 53.4, 46.2, 42.1, 36.7, 34.7, 34.3, 29.1, 29.0, 22.8; MS (AP+) C₂₆H₃₄N₂O₃ (MH+).

### Compound 52

¹H-NMR (500 MHZ, CDCl₃): 1.60-1.83 (3H, m), 2.10-2.20 (2H, m), 2.50-2.82 (12H, m), 3.55-3.59 (2H, m), 3.69 (2H, s), 3.84 (3H, s), 5.12 (1H, brs), 6.63 (1H, dd, J=2.8, 8.2 Hz), 7.08 (1H, d, J=2.8 Hz), 7.18-7.32 (5H, m), 7.40 (1H, d, J=8.2 Hz), 11.42 (1H, brs); ¹³C-NMR (500 MHZ, CDCl₃): 160.0, 145.7, 141.3, 139.2, 134.8, 129.2, 128.8, 126.7, 126.7, 120.9, 115.9, 111.8, 57.1, 56.3, 53.4, 43.2, 42.2, 38.8, 36.8, 35.1, 29.0, 22.8; MS (AP+) C₂₆H₃₄N₂O₃ (MH+).

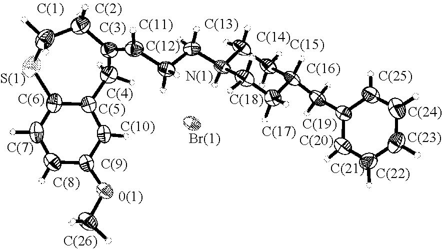

### REFERENCES

1. Wehrens, X. H. T.; Lehnart, S. E.; Reiken, S. R.; Deng, S.-X.; Vest, J. A.; Cervantes, D.; Coromilas, J. Landry, D. W.; Marks, A. R. Science 2004, 304, 292-296.
2. Kurti, L.; Czako, B. Strategic Applications of Named Reactions in Organic Synthesis; Elsevier Inc.: Amsterdam, 2005; pp 396-397.
3. (a). Grunewald, G. L.; Dahanukar, V. H.; Ching, P.; Criscione, K. R. J. Med. Chem. 1996, 39, 3539-3546. (b). Kaye, P. T.; Mphahlele, M. J. Synth. Commun. 1995, 25, 1495-1509.
4. Li, J.-T.; Li, H.-Y.; Li, H.-Z.; Xiao, L.-W. J. Chem. Res. 2004, 394-395.
5. Ponticello, G. S.; Freedman, M. B. J. Org. Chem. 1988, 53, 9-13.
6. Kumar, A.; Ner, D. H.; Dike, S. Indian J. of Chem. 1992, 31B, 803-809.
7. Miyata, O.; Shinada, T.; Ninomiya, I.; Naito, T.; Date, T.; Okamura, K.; Inagaki, S. J. Org. Chem., 1991, 56, 6556-6564.
8. Adam, G.; Andrieux, J.; Plat, M. Tetrahedron 1982, 38, 2403-2410.
9. (a) Hua, D. H.; Wu, S.; Narasimha, B.; Katsuhira, T.; Bravo, A. A. J. Org. Chem. 1990, 55, 3682-3684. (b) Ahn, Y.; Cardenas, G. I.; Yang, J.; Romo, D. Org. Lett. 2001, 3, 751-754.
10. Noda, Y.; Watanabe, M.; Helv. Chim. Acta. 2002, 85, 3473-3477.

### Example 2

### Calstabin 2 (FKBP12.6) Assay

PKA-phosphorylated cardiac sarcoplasmic reticulum (CSR) (approximately 20 μg) is incubated with 250 nM calstabin2 (FKBP12.6) in 100 μl binding buffer (10 mM imidazole, 300 mM sucrose, pH 7.4) with a known concentration of the test compound. Calstabin2 is added as the last reagent in the reaction mixture. The binding is performed at room temperature for 30 minutes. After the binding reaction, the mixture is centrifuged for 10 minutes at 100,000 xg. The resulting pellet is washed 4 times in binding buffer. After each wash, the tubes are centrifuged at 100,000 xg for 10 minutes. After the final wash, the supernatant is aspirated off 20 μl of 2×SDS-PAGE loading buffer is added to each pellet, and the pellet is resuspended. The mixture is heated to 90° C. for 4 minutes, and the proteins are analysed by 15% SDS-PAGE. Calstabin2 binding was detected with an αFKBP (1:2000) primary antibody and an IR-labeled anti-rabbit IgG from LiCor Biosciences as the secondary antibody. The blot was developed with a LiCor Odyssey system.

| Compound | Binding |
|---|---|
| 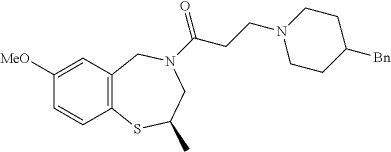 | 100 nM |
| 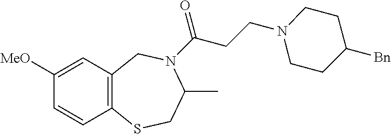 | 100 nM |
| 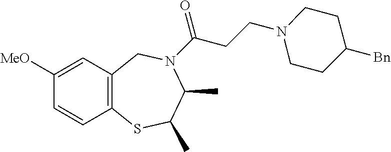 | 100 nM |
| 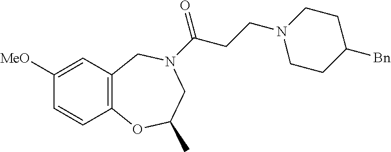 | 100 nM |
| 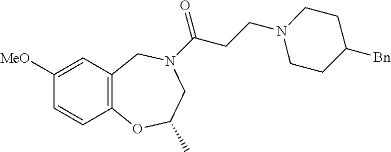 | 100 nM |
| 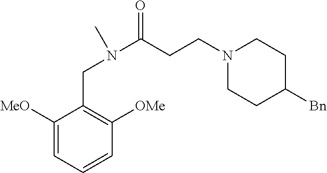 | 1 μM |
| 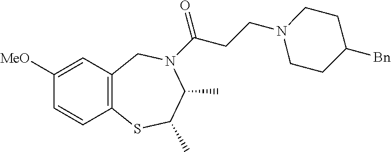 | 1 μM |
| 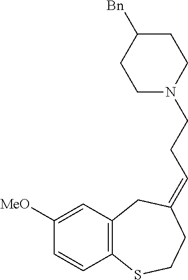 | 1 μM |
| 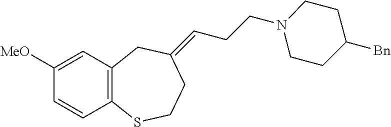 | 100 nM |

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. A compound of formula: 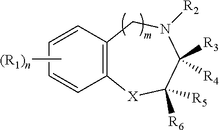
wherein
X is S or O;
m is 0, 1, or 2;
n is an integer between 0 and 4, inclusive;
R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —CN; —SCN; —SR_{B}; —SOR_{B}; —SO₂R_{B}; —NO₂; —N₃; —N(R_{B})₂; —NHC(═O)R_{B}; —NR_{B}C(═O)N(R_{B})₂; —OC(═O)OR_{B}; —OC(═O)R_{B}; —OC(═O)N(R_{B})₂; —NR_{B}C(═O)OR_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —CN; —SCN; —SR_{C}; —SOR_{C}; —SO₂R_{C}; —NO₂; —N₃; —N(R_{C})₂; —NHC(═O)R_{C}; —NR_{C}C(═O)N(R_{C})₂; —OC(═O)OR_{C}; —OC(═O)R_{C}; —OC(═O)N(R_{C})₂; —NR_{C}C(═O)OR_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₄ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; —C(═O)R_{D}; —CO₂R_{D}; —CN; —SCN; —SR_{D}; —SOR_{D}; —SO₂R_{D}; —NO₂; —N₃; —N(R_{D})₂; —NHC(═O)R_{D}; —NR_{D}C(═O)N(R_{D})₂; —OC(═O)OR_{D}; —OC(═O)R_{D}; —OC(═O)N(R_{D})₂; —NR_{D}C(═O)OR_{D}; or —C(R_{D})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₅ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{E}; —C(═O)R_{E}; —CO₂R_{E}; —CN; —SCN; —SR_{E}; —SOR_{E}; —SO₂R_{E}; —NO₂; —N₃; —N(R_{E})₂; —NHC(═O)R_{E}; —NR_{E}C(═O)N(R_{E})₂; —OC(═O)OR_{E}; —OC(═O)R_{E}; —OC(═O)N(R_{E})₂; —NR_{E}C(═O)OR_{E}; or —C(R_{E})₃; wherein each occurrence of R_{E} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₆ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{F}; —C(═O)R_{F}; —CO₂R_{F}; —CN; —SCN; —SOR_{F}; —SO₂R_{F}; —NO₂; —N₃; —N(R_{F})₂; —NHC(═O)R_{F}; —NR_{F}C(═O)N(R_{F})₂; —OC(═O)OR_{F}; —OC(═O)R_{F}; —OC(═O)N(R_{F})₂; —NR_{F}C(═O)OR_{F}; or —C(R_{F})₃; wherein each occurrence of R_{F} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

2. The compound of claim 1, wherein the compound is not JTV-519 of formula:

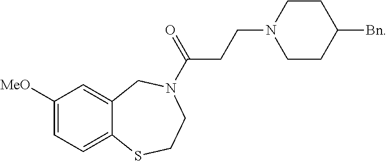

3. The compound of claim 1, wherein the compounds is not of the formula: 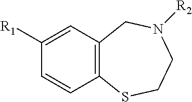
wherein R₁ is —OMe; and R₂ is —C(═O)R_{B} or —SO₂R_{B}.

4. The compound of claim 1, wherein X is O.

5. The compound of claim 1, wherein X is S.

6. The compound of claim 1, wherein m is 1.

7. (canceled)

8. The compound of claim 1, wherein n is 1.

9. (canceled)

10. The compound of claim 1, wherein R₁ is —OR_{A}.

11. The compound of claim 1, wherein R₁ is —OR_{A}, wherein R_{A} is C₁-C₆ alkyl.

12. The compound of claim 1, wherein R₁ is —OMe.

13.-16. (canceled)

17. The compound of claim 1, wherein R₂ is acyl.

18. The compound of claim 1, wherein R₂ is —(CO)—R_{B}.

19. (canceled)

20. The compound of claim 1, wherein R₂ is —(CO)—R_{B}, wherein R_{B} is cyclic or acyclic, substituted or unsubstituted heteroaliphatic.

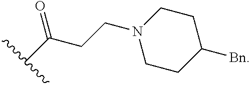

21. The compound of claim 1, wherein R₂ is

22.-29. (canceled)

30. The compound of claim 1, wherein at least one of R₃-R₆ is not hydrogen.

31. The compound of claim 1, wherein only one of R₃-R₆ is methyl, and the others are hydrogen.

32. The compound of claim 1, wherein only two of R₃-R₆ are methyl, and the others are hydrogen.

33.-35. (canceled)

36. The compound of claim 1 of formula:

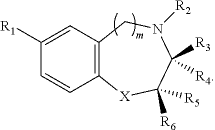

37.-42. (canceled)

43. The compound of claim 1 of one of the formulae:

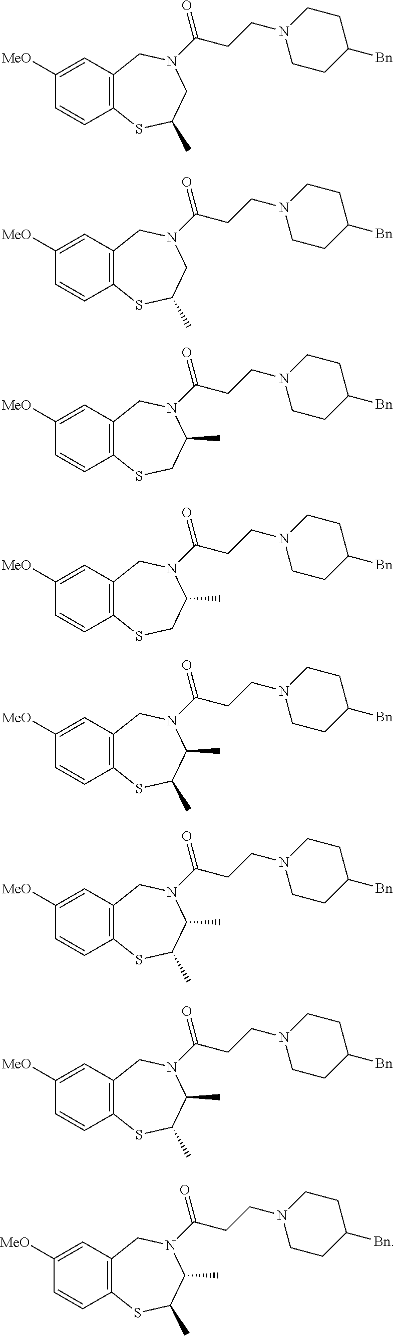

44. The compound of claim 1 of one of the formulae:

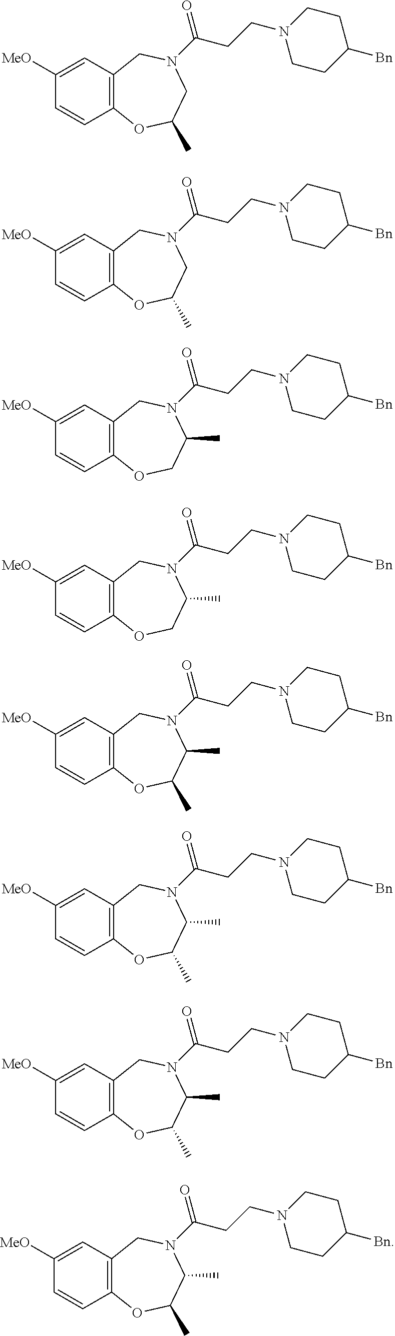

45.-46. (canceled)

47. A compound of formula: 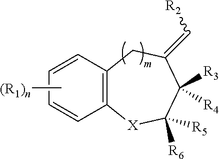
wherein
X is S or O;
m is 0, 1, or 2;
n is an integer between 0 and 4, inclusive;
R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —SR_{B}; —SOR_{B}; —SO₂R_{B}; —N(R_{B})₂; —NHC(═O)R_{B}; —NR_{B}C(═O)N(R_{B})₂; —OC(═O)OR_{B}; —OC(═O)R_{B}; —OC(═O)N(R_{B})₂; —NR_{B}C(═O)OR_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —CN; —SCN; —SR_{C}; —SOR_{C}; —SO₂R_{C}; —NO₂; —N₃; —N(R_{C})₂; —NHC(═O)R_{C}; —NR_{C}C(═O)N(R_{C})₂; —OC(═O)OR_{C}; —OC(═O)R_{C}; —OC(═O)N(R_{C})₂; —NR_{C}C(═O)OR_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₄ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; —C(═O)R_{D}; —CO₂R_{D}; —CN; —SCN; —SR_{C}; —SOR_{D}; —SO₂R_{D}; —NO₂; —N₃; —N(R_{D})₂; —NHC(═O)R_{D}; —NR_{D}C(═O)N(R_{D})₂; —OC(═O)OR_{D}; —OC(═O)R_{D}; —OC(═O)N(R_{D})₂; —NR_{D}C(═O)OR_{D}; or —C(R_{D})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₅ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{E}; —C(═O)R_{E}; —CO₂R_{E}; —CN; —SCN; —SR_{E}; —SOR_{E}; —SO₂R_{E}; —NO₂; —N₃; —N(R_{E})₂; —NHC(═O)R_{E}; —NR_{E}C(═O)N(R_{E})₂; —OC(═O)OR_{E}; —OC(═O)R_{E}; —OC(═O)N(R_{E})₂; —NR_{E}C(═O)OR_{E}; or —C(R_{E})₃; wherein each occurrence of R_{E} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₆ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{F}; —C(═O)R_{F}; —CO₂R^{F}; —CN; —SCN; —SOR_{F}; —SO₂R_{F}; —NO₂; —N₃; —N(R_{F})₂; —NHC(═O)R_{F}; —NR_{F}C(═O)N(R_{F})₂; —OC(═O)OR_{F}; —OC(═O)R_{F}; —OC(═O)N(R_{F})₂; —NR_{F}C(═O)OR_{F}; or —C(R_{F})₃; wherein each occurrence of R_{F} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

48.-79. (canceled)

80. A pharmaceutical composition comprising a compound of claim 1 and a pharmaceutically acceptable excipient.

81.-85. (canceled)

86. A method of treating cardiac disease comprising:
administering a therapeutically effective amount of a compound of claim 1 to a subject with cardiac disease or susceptible to cardiac disease.

87.-102. (canceled)
